(19) United States
(12) Patent Application Publication
(10) Pub. No.: US 2022/0105121 A1
(43) Pub. Date: Apr. 7, 2022

(54) COMPOSITIONS AND METHODS FOR INHIBITING INOSINE MONOPHOSPHATE DEHYDROGENASE

(71) Applicant: Clear Creek Bio, Inc., Cambridge, MA (US)

(72) Inventors: Vikram S. Kumar, Boston, MA (US); David P. Hesson, Malvern, PA (US)

(21) Appl. No.: 17/426,670

(22) PCT Filed: Feb. 5, 2020
(86) PCT No.: PCT/US2020/016689

§ 371 Date: Jul. 29, 2021

Related U.S. Application Data
(60) Provisional application No. 62/803,027, filed on Feb. 8, 2019.

(51) Int. Cl. A61K 31/7056; A61P 35/02

(52) U.S. Cl. CPC A61K 31/7056; A61P 35/02

(57) ABSTRACT

The invention provides compositions and methods for treating conditions in a subject by providing an inhibitor of inosine phosphate dehydrogenase that promotes decreased levels of guanosine triphosphate and/or increased levels of hypoxanthine.

### CROSS-REFERENCE TO RELATED APPLICATIONS

This application claims the benefit of, and priority to, U.S. Provisional Patent Application No. 62/803,027, filed Feb. 8, 2019, the contents of which are incorporated by reference.

### FIELD OF THE INVENTION

The invention generally relates to therapeutic compositions and methods.

### BACKGROUND

Proliferating cancer cells show substantially different metabolic needs compared to normal differentiated cells, as they require additional nutrients to support their high rates of proliferation. Success in targeting cancer cell metabolism will materialize from an improved understanding of exactly how cells control and consume nutrients into pathways that are essential for biosynthesis. As all cancer cells rely on this alteration in metabolism, these altered pathways represent strong therapeutic targets. However, discovering a therapeutic window between normal proliferating cells and cancer cells remains a major challenge because the metabolic requirements of these cells are similar. Thus, only a few molecules which target metabolic pathways have been established as a form of cancer treatment.

One enzyme that is the target of multiple candidates for antineoplastic therapeutics is inosine monophosphate dehydrogenase (IMPDH). IMPDH catalyzes the rate-limiting reaction in de novo synthesis of guanine nucleotides. Cancer cells require a robust supply of nucleotides to support rapid genome replication, and inhibition of IMPDH blocks proliferation of cancer cells. However, healthy cells also depend on de novo purine synthesis for certain essential functions, so IMPDH inhibitors can harm them as well. For example, the IMPDH inhibitor tiazofurin produces a variety of adverse events at high doses. In addition, tiazofurin is rapidly cleared from the body, making the drug difficult to administer effectively other than by continuous intravenous infusion. Therefore, IMPDH inhibitors, such as tiazofurin and its analogs, are not currently considered in the treatment of cancer.

### SUMMARY

The invention recognizes that IMPDH inhibitors, such as tiazofurin, have failed in the past because they have not been dosed to achieve optimal enzyme inhibition. Methods of the invention solve that problem by administering IMPDH inhibitors to a patient to achieve nearly complete inhibition of IMPDH for a sustained yet finite period. The period of sustained IMDPH inhibition is followed by a “drug holiday” period in which the inhibitor is withheld from the patient, allowing healthy cells to recover. By choosing appropriate durations for the inhibitory and recovery periods, methods of the invention allow optimal dosing of an IMPDH inhibitor to kill cancer cells without causing harmful and toxic side effects to patients.

Another insight of the invention is that levels of specific metabolites can serve as highly sensitive markers of target engagement by IMPDH inhibitors. In particular, decreases in guanosine triphosphate (GTP) levels relative to a reference level are used to determine engagement of an active pharmaceutical agent (API) with IMPDH. By monitoring levels of GTP, oral doses of IMPDH inhibitors, such as tiazofurin, that achieve nearly complete IMPDH inhibition can be provided at reasonable intervals, such as three times per day. Consequently, the methods and compositions of the invention not only improve the therapeutic efficacy of IMPDH inhibitors, they also support and monitor patient adherence by providing biomarkers that correlate with dosing regimens and can serve as positive feedback to patients and their care teams.

The methods of the invention also allow dosing of IMPDH inhibitors, such as tiazofurin, to be tailored to an individual patient. Metabolism of drugs varies among patients, so individuals who have received the same dose of the same drug may differ in their response. However, levels of GTP and hypoxanthine are universal indicators of purine metabolism across all patients. Thus, by determining dosage based on the effect of an IMPDH inhibitor, such as tiazofurin, in a given patient, the methods permit physicians to design customized dosing regimens that are optimized for individual patients.

The methods and compositions of the invention are useful to treat any condition associated with IMPDH activity that is altered, unregulated, or essential for the survival of a class of cells, such as cancer cells. In particular, the methods and compositions are useful for treating cancer, such as leukemia.

In an aspect, the invention provides compositions containing an inhibitor of inosine monophosphate dehydrogenase in a therapeutically effective amount that lowers or maintains in a subject a level of guanosine triphosphate (GTP) compared to a reference GTP level for a period.

The period may be about 24 hours, about 36 hours, about 48 hours, about 60 hours, about 72 hours, about 84 hours, about 96 hours, about 108 hours, about 120 hours, about 6 days, about 7 days, about 8 days, about 9 days, about 10 days, about 12 days, about 2 weeks, about 3 weeks, or about 4 weeks.

The composition may contain an inhibitor of IMPDH in a therapeutically effective amount that lowers or maintains a level of guanosine triphosphate (GTP) in a subject that is less than about 20%, less than about 15%, less than about 10%, less than about 5%, from about 5% to less than about 20%, from about 10% to less than about 20%, from about 5% to about 15%, or from about 5% to about 10% of a reference level.

The reference GTP level may be a level of GTP in a subject prior to administration of the composition to the subject. The reference GTP level may be a level of GTP in a subject that has not received the composition. In some embodiments, the reference GTP level is a level of GTP in the same subject prior to administration of the composition to the subject, such as a baseline GTP level of the subject. The reference level of may be an average level of GTP in a group of subjects prior to administration of the composition to the subjects or an average level of GTP in subjects that have not received the composition, such as an average baseline GTP level of a group of subjects.

The inhibitor of inosine monophosphate dehydrogenase may be tiazofurin, mycophenolic acid, ribavirin, mizoribine, selenazofurin, taribavirin, or an analog, derivative, prodrug, micellar formulation, sustained release formulation, or salt of any of the aforementioned compounds. The therapeutically effective amount of the inhibitor may be from about 200 mg/m² to about 12,000 mg/m², from about 500 mg/m² to about 10,000 mg/m², from about 1000 mg/m² to about 8000 mg/m², from about 2000 mg/m² to about 5000 mg/m², from about 3000 mg/m² to about 4000 mg/m², about 1000 mg/m², about 2000 mg/m², about 3000 mg/m², about 3300 mg/m², about 4000 mg/m², about 5000 mg/m², about 6000 mg/m², about 7000 mg/m², about 7600 mg/m², or about 8000 mg/m². The therapeutically effective amount of the inhibitor may be from about 0.8 g to about 12 g, from about 1 g to about 10 g, from about 2 g to about 6 g, from about 3 g to about 5 g, about 0.6 g, about 0.8 g, about 1 g, about 2 g, about 3 g, about 4 g, about 5 g, about 6 g, about 8 g, about 10 g, about 12 g, or about 15 g

The composition may be formulated as a single-unit dosage. The composition may be formulated as divided dosages.

The composition may be formulated for administration intravenously, orally, enterally, parenterally, dermally, transdermally, by injection, subcutaneously, pulmonarily, or with or on an implantable medical device. In some embodiments, the composition is formulated for intravenous administration. In some embodiments, the composition is formulated for parenteral administration.

The composition may contain a xanthine oxidase inhibitor. The xanthine oxidase inhibitor may be allopurinol, oxypurinol, tisopurine, topiroxostat, phytic acid, or myoinositol. The xanthine oxidase inhibitor may be present in a therapeutically effective amount that maintains a hypoxanthine level that is from about 500% to about 1500%, from about 600% to about 1400%, from about 700% to about 1300%, from about 800% to about 1200%, or from about 900% to about 1100% of a reference hypoxanthine level.

The reference hypoxanthine level may be a level of hypoxanthine in a subject prior to administration of the composition to the subject or a level of hypoxanthine in a subject that has not received the composition. In some embodiments, the reference hypoxanthine level is a level of hypoxanthine in the same subject prior to administration of the composition to the subject, such as a baseline hypoxanthine level of the subject. The reference level of may be an average level of hypoxanthine in a group of subjects prior to administration of the composition to the subjects or an average level of hypoxanthine in subjects that have not received the composition, such as an average baseline hypoxanthine level of a group of subjects.

The composition may be useful treat a condition in the subject. The condition may be cancer. The condition may be acute myeloid leukemia (AML), acute lymphoblastic leukemia (ALL), adult T cell leukemia/lymphoma (ATLL), bladder cancer, breast cancer, such as triple negative breast cancer (TNBC), glioma, head and neck cancer, leukemia, such as AML, lung cancer, such as small cell lung cancer or non-small cell lung cancer, lymphoma, multiple myeloma, neuroblastoma, osteosarcoma, ovarian cancer, prostate cancer, brain cancer, or renal cell cancer.

The composition may be effective for treating a condition, such as any of the conditions described above, in the subject.

In another aspect, the invention provides oral pharmaceutical compositions comprising from about 10 mg to about 1,500 mg of tiazofurin or an analog, derivative, prodrug, micellar formulation, sustained release formulation, or salt thereof.

The oral compositions may contain from about 20 mg to about 1,200 mg, from about 50 mg to about 1,000 mg, from about 100 mg to about 500 mg, about 10 mg, about 20 mg, about 50 mg, about 100 mg, about 200 mg, about 300 mg, about 400 mg, or about 500 mg of tiazofurin or an analog, derivative, prodrug, micellar formulation, sustained release formulation, or salt thereof.

The composition may be formulated as a single-unit dosage. The composition may be formulated as divided dosages.

In another aspect, the invention provides a dosage containing from about 0.2 g to about 5 g of orally deliverable tiazofurin or an analog, derivative, prodrug, micellar formulation, sustained release formulation, or salt thereof.

The dosage may contain from about 0.3 g to about 3 g, from about 0.4 g to about 2 g, from about 0.4 g to about 2 g, about 0.2 g, about 0.3 g, about 0.4 g, about 0.5 g, about 0.6 g, about 0.8 g, about 1 g, or about 2 g of orally deliverable tiazofurin or the analog, derivative, prodrug, micellar formulation, sustained release formulation, or salt thereof.

The dosage may be in a single physical unit. The dosage may be divided among multiple physical units.

Administration of the dosage to a subject multiple times in a 24-hour period may provide tiazofurin or an analog, derivative, prodrug, micellar formulation, sustained release formulation, or salt thereof in a therapeutically effective amount that lowers or maintains in the subject a guanosine triphosphate (GTP) compared to a reference GTP level for a period. The dosage may be administered two times, three times, four times, five times, six times, eight times, or more in a 24-hour period to provide a therapeutically effective amount that lowers or maintains in the subject a guanosine triphosphate (GTP) compared to a reference GTP level for a period.

The period may be about 24 hours, about 36 hours, about 48 hours, about 60 hours, about 72 hours, about 84 hours, about 96 hours, about 108 hours, about 120 hours, about 6 days, about 7 days, about 8 days, about 9 days, about 10 days, about 12 days, about 2 weeks, about 3 weeks, or about 4 weeks.

The therapeutically effective amount may lower or maintain a level of guanosine triphosphate (GTP) in a subject that is less than about 20%, less than about 15%, less than about 10%, less than about 5%, from about 5% to less than about 20%, from about 10% to less than about 20%, from about 5% to about 15%, or from about 5% to about 10% of a reference level.

The reference GTP level may be any of those described above. For example, the reference GTP level may be a level of GTP in a subject prior to administration of the composition to the subject.

In another aspect, the invention provides a divided dosage containing from about 0.6 g to about 15 g of orally deliverable tiazofurin or an analog, derivative, prodrug, micellar formulation, sustained release formulation, or salt thereof.

The divided dosage may contain from about 0.8 g to about 12 g, from about 1 g to about 10 g, from about 2 g to about 6 g, from about 3 g to about 5 g, about 0.6 g, about 0.8 g, about 1 g, about 2 g, about 3 g, about 4 g, about 5 g, about 6 g, about 8 g, about 10 g, about 12 g, or about 15 g of orally deliverable tiazofurin or an analog, derivative, prodrug, micellar formulation, sustained release formulation, or salt thereof.

Administration of the divided dosage to a subject in a 24-hour period may provide tiazofurin or an analog, derivative, prodrug, micellar formulation, sustained release formulation, or salt thereof in a therapeutically effective amount that lowers or maintains in the subject a guanosine triphosphate (GTP) compared to a reference GTP level for a period. The dosage may be administered two times, three times, four times, five times, six times, or more in a 24-hour period to provide a therapeutically effective amount that lowers or maintains in the subject a guanosine triphosphate (GTP) compared to a reference GTP level for a period.

The period may be about 24 hours, about 36 hours, about 48 hours, about 60 hours, about 72 hours, about 84 hours, about 96 hours, about 108 hours, about 120 hours, about 6 days, about 7 days, about 8 days, about 9 days, about 10 days, about 12 days, about 2 weeks, about 3 weeks, or about 4 weeks.

The therapeutically effective amount may lower or maintain a level of guanosine triphosphate (GTP) in a subject that is less than about 20%, less than about 15%, less than about 10%, less than about 5%, from about 5% to less than about 20%, from about 10% to less than about 20%, from about 5% to about 15%, or from about 5% to about 10% of a reference level.

The reference GTP level may be any of those described above. For example, the reference GTP level may be a level of GTP in a subject prior to administration of the composition to the subject.

The divided dosage may include 2 parts, 3 parts, 4 parts, 5 parts, 6 parts, or more. Each of the parts may be administered at a different point in the 24-hour period.

In another aspect, the invention provides methods of treating a condition in a subject. The methods include a first phase in which one or more doses of a composition containing an inhibitor of inosine monophosphate dehydrogenase are provided to a subject in a therapeutically effective amount that lowers or maintains in the subject a guanosine triphosphate (GTP) level compared to a reference GTP level for a first period and a second phase in which the composition is not provided for a second period.

The one or more doses provided during the first phase may lower or maintain a guanosine triphosphate (GTP) level in a subject that is less than about 20%, less than about 15%, less than about 10%, less than about 5%, from about 5% to less than about 20%, from about 10% to less than about 20%, from about 5% to about 15%, or from about 5% to about 10% of a reference level.

The reference GTP level may be a level of GTP in a subject prior to administration of the composition to the subject. The reference GTP level may be a level of GTP in a subject that has not received the composition. In some embodiments, the reference GTP level is a level of GTP in the same subject prior to administration of the composition to the subject, such as a baseline GTP level of the subject. The reference level of may be an average level of GTP in a group of subjects prior to administration of the composition to the subjects or an average level of GTP in subjects that have not received the composition, such as an average baseline GTP level of a group of subjects.

The first period may be at least 24 hours, at least 36 hours, at least 48 hours, at least 60 hours, at least 72 hours, at least 84 hours, at least 96 hours, at least 108 hours, at least 120 hours, at least 6 days, at least 7 days, at least 8 days, at least 9 days, at least 10 days, at least 12 days, at least 2 weeks, at least 3 weeks, at least 4 weeks, from about 48 hours to about 72 hours, from about 48 hours to about 96 hours, from about 48 hours to about 120 hours, from about 72 hours to about 96 hours, or from about 72 hours to about 120 hours, from about 2 days to about 7 days, from about 3 days to about 7 days, from about 4 days to about 7 days, from about 5 days to about 7 days, or from about 6 days to about 7 days.

During the first phase, the doses may be provided about every 24 hours, about every 12 hours, about every 8 hours, about every 6 hours, about every 4 hours, about every 3 hours, about every 2 hours, or about every 1 hour.

The doses may be provided orally, intravenously, enterally, parenterally, dermally, transdermally, by injection, subcutaneously, pulmonarily, or with or on an implantable medical device.

The inhibitor of inosine monophosphate dehydrogenase may be tiazofurin, mycophenolic acid, ribavirin, mizoribine, selenazofurin, taribavirin, or an analog, derivative, prodrug, micellar formulation, sustained release formulation, or salt of any of the aforementioned compounds.

The method may include providing a xanthine oxidase inhibitor. The xanthine oxidase inhibitor may be allopurinol, oxypurinol, tisopurine, topiroxostat, phytic acid, or myoinositol. The xanthine oxidase inhibitor may be provided during the first phase, second phase, or both.

The method may include determining the GTP level in the subject at an end of the first phase and determining a duration of the second phase based on the GTP level in the subject at the end of the first phase.

The method may include evaluating renal function in the subject prior to initiating the first phase. For example, evaluating renal function may include determining that the subject has a glomerular filtration rate of at least about 10%, at least about 20%, at least about 30%, at least about 40%, at least about 50%, at least about 60%, at least about 70%, at least about 80%, or at least about 90% of a reference glomerular filtration rate.

The reference glomerular filtration rate may be a predicted glomerular filtration rate for the subject. The predicted glomerular filtration rate may be based on one or more properties of the subject, such as age, sex, weight, or medical condition.

Evaluation of renal function may include analysis of a level of an analyte in a body fluid of the subject. The analyte may be creatinine, ethylenediaminetetraacetic acid (EDTA), inulin, pentetic acid (DTPA), protein, sinistrin, or urea. The body fluid may be urine, blood, plasma, or serum.

The method may include determining the one or more doses based on the subject's renal function. For example, the one or more doses may be reduced relative to a reference dose when the subject has a decreased glomerular filtration rate compared to a reference glomerular filtration rate.

The second period may be at least 24 hours, at least 36 hours, at least 48 hours, at least 60 hours, at least 72 hours, at least 84 hours, at least 96 hours, at least 108 hours, at least 120 hours, at least 6 days, at least 7 days, at least 8 days, at least 9 days, at least 10 days, at least 12 days, at least 2 weeks, at least 3 weeks, at least 4 weeks, from about 48 hours to about 72 hours, from about 48 hours to about 96 hours, from about 48 hours to about 120 hours, from about 72 hours to about 96 hours, or from about 72 hours to about 120 hours, from about 2 days to about 7 days, from about 3 days to about 7 days, from about 4 days to about 7 days, from about 5 days to about 7 days, or from about 6 days to about 7 days.

The method may include a third phase in which one or more doses of a composition containing an inhibitor of inosine monophosphate dehydrogenase are provided to a subject in a therapeutically effective amount that lowers or maintains in the subject a guanosine triphosphate (GTP) level below 10% of a reference GTP level for a third period.

The third period may have any duration described above for the first period.

The third phase may follow immediately after the second phase.

The inhibitor of inosine monophosphate dehydrogenase may be tiazofurin or an analog, derivative, prodrug, micellar formulation, sustained release formulation, or salt thereof.

The composition may be provided in multiple doses to deliver from about 1 g to about 5 g of tiazofurin or the analog, derivative, prodrug, micellar formulation, sustained release formulation, or salt thereof to the subject in each 24-hour period during the first phase. The composition may be provided in multiple doses to deliver about 3 g of tiazofurin or the analog, derivative, prodrug, micellar formulation, sustained release formulation, or salt thereof to the subject in each 24-hour period during the first phase. The composition may be provided in 2, 3, 4, 5, 6, 8, or more doses in each 24-hour period.

The doses may be provided orally, intravenously, enterally, parenterally, dermally, transdermally, by injection, subcutaneously, pulmonarily, or with or on an implantable medical device.

The method may be useful for treating any condition, such as any of those described above, in the subject.

The method may include providing a composition containing a xanthine oxidase inhibitor. The xanthine oxidase inhibitor may be allopurinol, oxypurinol, tisopurine, topiroxostat, phytic acid, or myoinositol. The xanthine oxidase inhibitor may be provided in the same composition as the inosine monophosphate dehydrogenase inhibitor. The xanthine oxidase inhibitor may be provided in a different composition from the inosine monophosphate dehydrogenase inhibitor.

The composition containing the xanthine oxidase inhibitor may be provided in a therapeutically effective amount that maintains a hypoxanthine level that is from about 500% to about 1500%, from about 600% to about 1400%, from about 700% to about 1300%, from about 800% to about 1200%, or from about 900% to about 1100% of a reference hypoxanthine level.

The reference hypoxanthine level may be a level of hypoxanthine in a subject prior to administration of the composition to the subject or a level of hypoxanthine in a subject that has not received the composition. In some embodiments, the reference hypoxanthine level is a level of hypoxanthine in the same subject prior to administration of the composition to the subject. The reference level of may be an average level of hypoxanthine in a group of subjects prior to administration of the composition to the subjects or an average level of hypoxanthine in subjects that have not received the composition.

In another aspect, the invention provides methods of treating a condition in a subject. The methods include a first phase in which one or more doses of tiazofurin or an analog, derivative, prodrug, micellar formulation, sustained release formulation, or salt thereof are provided orally in each 24-hour period.

The duration of the first phase may be from about 24 hours to about 48 hours, from about 24 hours to about 72 hours, from about 24 hours to about 96 hours, from about 24 hours to about 120 hours, from about 48 hours to about 72 hours, from about 48 hours to about 96 hours, from about 48 hours to about 120 hours, from about 72 hours to about 96 hours, from about 72 hours to about 120 hours, from about 96 hours to about 120 hours, about 24 hours, about 36 hours, about 48 hours, about 60 hours, about 72 hours, about 84 hours, about 96 hours, about 108 hours, about 120 hours, about 6 days, about 7 days, about 8 days, about 9 days, about 10 days, about 11 days, about 12 days, about 2 weeks about 3 weeks, or about 4 weeks.

The one or more doses may deliver from about 0.2 g to about 15 g, from about 0.5 g to about 15 g, from about 1 g to about 15 g, from about 0.2 g to about 10 g, from about 0.5 g to about 10 g, from about 1 g to about 10 g, from about 0.2 g to about 8 g, from about 0.5 g to about 8 g, from about 1 g to about 8 g, from about 0.2 g to about 5 g, from about 0.4 g to about 5 g, from about 1 g to about 5 g, about 0.2 g, about 0.5 g, about 1 g, about 2 g, about 3 g, about 4 g, about 5 g, about 8 g, about 10 g, or about 15 g tiazofurin or an analog, derivative, prodrug, micellar formulation, sustained release formulation, or salt thereof in each 24-hour period.

The method may include providing 1, 2, 3, 4, 5, 6, 8, or more oral doses in each 24-hour period.

The method may include a second phase in which no doses of tiazofurin or the analog, derivative, prodrug, micellar formulation, sustained release formulation, or salt thereof are provided to the subject.

The method may be useful for treating any condition, such as any of those described above, in the subject.

The method may include providing a composition containing a xanthine oxidase inhibitor, such as any of those described above. The composition containing the xanthine oxidase inhibitor may be provided in a therapeutically effective amount that maintains a hypoxanthine level that is from about 500% to about 1500%, from about 600% to about 1400%, from about 700% to about 1300%, from about 800% to about 1200%, or from about 900% to about 1100% of a reference hypoxanthine level. The reference hypoxanthine level may be any such level described above.

In another aspect, the invention provides methods of treating a condition in a subject. The methods include providing orally to a subject one or more doses of tiazofurin or an analog, derivative, prodrug, micellar formulation, sustained release formulation, or salt thereof in a 24-hour period.

The one or more doses may deliver from about 0.2 g to about 15 g, from about 0.5 g to about 15 g, from about 1 g to about 15 g, from about 0.2 g to about 10 g, from about 0.5 g to about 10 g, from about 1 g to about 10 g, from about 0.2 g to about 8 g, from about 0.5 g to about 8 g, from about 1 g to about 8 g, from about 0.2 g to about 5 g, from about 0.4 g to about 5 g, from about 1 g to about 5 g, about 0.2 g, about 0.5 g, about 1 g, about 2 g, about 3 g, about 4 g, about 5 g, about 8 g, about 10 g, or about 15 g tiazofurin or an analog, derivative, prodrug, micellar formulation, sustained release formulation, or salt thereof in the 24-hour period.

The method may involve providing the one or more doses repeatedly over multiple 24-hour periods or fractions thereof. For example, the method may be repeated for about 36 hours, 48 hours, about 60 hours, about 72 hours, about 84 hours, about 96 hours, or about 120 hours.

The method include, after providing the one or more doses, withholding doses of tiazofurin or the analog, derivative, prodrug, micellar formulation, sustained release formulation, or salt thereof. The withholding period may be about 12 hours, about 24 hours, about 36 hours, about 48 hours, about 60 hours, or about 72 hours.

The method may be useful for treating any condition, such as any of those described above, in the subject.

The method may include providing a composition containing a xanthine oxidase inhibitor, such as any of those described above. The composition containing the xanthine oxidase inhibitor may be provided in a therapeutically effective amount that maintains a hypoxanthine level that is from about 500% to about 1500%, from about 600% to about 1400%, from about 700% to about 1300%, from about 800% to about 1200%, or from about 900% to about 1100% of a reference hypoxanthine level. The reference hypoxanthine level may be any such level described above.

In another aspect, the invention provides methods of treating a condition in a subject by providing to the subject a composition containing an inhibitor of inosine monophosphate dehydrogenase in a therapeutically effective amount that maintains in a subject having a condition a level of guanosine triphosphate (GTP) in a subject that is less than about 20% of a reference GTP level.

The reference GTP level may be any suitable reference GTP level, such as those described above.

The composition containing the IMPDH inhibitor may be provided in a therapeutically effective amount that maintains a level of guanosine triphosphate (GTP) in a subject that is less than about 15%, less than about 10%, less than about 5%, from about 5% to less than about 20%, from about 10% to less than about 20%, from about 5% to about 15%, or from about 5% to about 10% of a reference level.

The inhibitor of inosine monophosphate dehydrogenase may be tiazofurin, mycophenolic acid, ribavirin, mizoribine, selenazofurin, taribavirin, or an analog, derivative, prodrug, micellar formulation, sustained release formulation, or salt of any of the aforementioned compounds.

The therapeutically effective amount of the inhibitor may be from about 200 mg/m² to about 12,000 mg/m², from about 500 mg/m² to about 10,000 mg/m², from about 1000 mg/m² to about 8000 mg/m², from about 2000 mg/m² to about 5000 mg/m², from about 3000 mg/m² to about 4000 mg/m², about 1000 mg/m², about 2000 mg/m², about 3000 mg/m², about 3300 mg/m², about 4000 mg/m², about 5000 mg/m², about 6000 mg/m², about 7000 mg/m², about 7600 mg/m², or about 8000 mg/m². The therapeutically effective amount of the inhibitor may be from about 0.8 g to about 12 g, from about 1 g to about 10 g, from about 2 g to about 6 g, from about 3 g to about 5 g, about 0.6 g, about 0.8 g, about 1 g, about 2 g, about 3 g, about 4 g, about 5 g, about 6 g, about 8 g, about 10 g, about 12 g, or about 15 g.

The therapeutically effective amount may be provided as a single unit dosage. The therapeutically effective amount may be provided as divided dosages.

The composition may be provided intravenously, orally, enterally, parenterally, dermally, transdermally, by injection, subcutaneously, pulmonarily, or with or on an implantable medical device. In some embodiments, the composition is provided intravenously. In some embodiments, the composition is provided parenterally.

The method may include providing a composition containing a xanthine oxidase inhibitor, such as any of those described above. The composition containing the xanthine oxidase inhibitor may be provided in a therapeutically effective amount that maintains a hypoxanthine level that is from about 500% to about 1500%, from about 600% to about 1400%, from about 700% to about 1300%, from about 800% to about 1200%, or from about 900% to about 1100% of a reference hypoxanthine level. The reference hypoxanthine level may be any such level described above.

The condition may be cancer, such as any of the cancers described above.

### BRIEF DESCRIPTION OF THE DRAWINGS

FIG. 1 is a graph of plasma tiazofurin levels in subjects after intravenous administration according to a method of the invention.

FIG. 2 is a graph of plasma tiazofurin levels in subjects after intravenous administration according to a method of the invention.

FIG. 3 is a graph of plasma tiazofurin levels in subjects after intravenous administration according to a method of the invention.

FIG. 4 is a graph of plasma tiazofurin levels in subjects after intravenous administration according to a method of the invention.

FIG. 5 is a graph of plasma tiazofurin levels in subjects after oral administration of a composition having 70% bioavailability according to a method of the invention.

FIG. 6 is a graph of plasma tiazofurin levels in subjects after oral administration of a composition having 80% bioavailability according to a method of the invention.

FIG. 7 is a graph of plasma tiazofurin levels in subjects after oral administration of a composition having 90% bioavailability according to a method of the invention.

FIG. 8 is a graph of plasma tiazofurin levels in subjects after oral administration of a composition having 70%, 80%, or 90% bioavailability according to a method of the invention.

FIG. 9 is a graph of plasma tiazofurin levels in subjects after oral administration of a composition having 70% bioavailability according to a method of the invention.

FIG. 10 is a graph of plasma tiazofurin levels in subjects after oral administration of a composition having 80% bioavailability according to a method of the invention.

FIG. 11 is a graph of plasma tiazofurin levels in subjects after oral administration of a composition having 90% bioavailability according to a method of the invention.

FIG. 12 is a graph of plasma tiazofurin levels in subjects after oral administration of a composition having 70%, 80%, or 90% bioavailability according to a method of the invention.

FIG. 13 is a graph of plasma tiazofurin levels in subjects after intravenous administration according to a method of the invention.

FIG. 14 is a graph of plasma tiazofurin levels in subjects after intravenous administration according to a method of the invention.

FIG. 15 is a graph of plasma tiazofurin levels in subjects after oral administration of a composition having 80% bioavailability according to a method of the invention.

FIG. 16 is a graph of plasma tiazofurin levels in subjects after oral administration of a composition having 80% bioavailability according to a method of the invention.

FIG. 17 is a graph of plasma tiazofurin levels in subjects after oral administration of compositions having 70%, 80%, or 90% bioavailability according to a method of the invention.

FIG. 18 is a graph of plasma tiazofurin levels in subjects after oral administration of a composition having 80% bioavailability according to a method of the invention.

FIG. 19 is a graph of plasma tiazofurin levels in subjects after oral administration of compositions having 70%, 80%, or 90% bioavailability according to a method of the invention.

FIG. 20 is a graph of plasma tiazofurin levels in subjects after intravenous administration according to a method of the invention.

FIG. 21 is a graph of plasma tiazofurin levels in subjects after oral administration of a composition having 80% bioavailability according to a method of the invention.

FIG. 22 is a graph of plasma tiazofurin levels in subjects after oral administration of a composition having 80% bioavailability according to a method of the invention.

FIG. 23 is a graph of plasma tiazofurin levels in subjects after oral administration of a composition having 80% bioavailability according to a method of the invention.

### DETAILED DESCRIPTION

Inhibitors of inosine monophosphate dehydrogenase (IMPDH) have been investigated as therapeutic agents to treat a variety of conditions, including cancer, viral and bacterial infections, and organ transplantation via immunosuppression. IMPDH activity is necessary for de novo synthesis of guanine nucleotides, such as guanosine triphosphate (GTP). Rapidly proliferating cells, such as cancer cells, immune cells, or infectious pathogens, have an increased demand for nucleotides, so inhibition of IMPDH curbs expansion of such cell types. However, guanine nucleotides are essential for cellular processes in other cell types as well. For example, GTP provides energy for protein synthesis and acts as an intracellular signaling molecule. Therefore, a challenge in using IMPDH inhibitors therapeutically is providing such agents in sufficient quantity to block proliferation of targeted cell populations without inflicting severe damage on healthy cells.

The invention solves the aforementioned problem by providing methods of monitoring target engagement of IMPDH inhibitors to allow precise dosing to optimize their therapeutic benefit. The invention recognizes that inhibition of IMPDH in a subject alters the levels of specific metabolites in fluid samples, such as blood samples, from the subject. By analyzing the level of a metabolite such as GTP or hypoxanthine, one can obtain a real-time indication of IMPDH inhibition and suppression of salvage in a given individual. The metabolic information can then be used to determine or adjust a dosing regimen of the inhibitor so that both the degree and duration of IMPDH inhibition are optimized for the individual.

### Indicators of Target Engagement for IMPDH Inhibitors

IMPDH catalyzes the first committed and rate-limiting step towards the de novo biosynthesis of guanine nucleotides. Some of the key reactions involved in synthesis of guanine nucleotides are shown below:

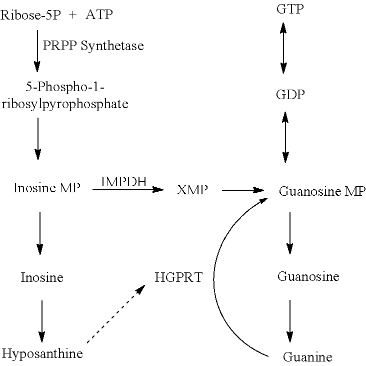

The multi-step conversion of ribose-5-phosphate (ribose-5P) to inosine monophosphate shown in the upper left portion of the diagram is required for all de novo purine synthesis. For synthesis of guanine nucleotides, such guanosine triphosphate (GTP) and deoxyguanosine triphosphate (dGTP), IMPDH catalyzes the nicotinamide adenine dinucleotide (NAM-dependent oxidation of inosine monophosphate (IMP) to xanthosine monophosphate (XMP). Guanosine monophosphate synthetase (GMPS) then converts XMP to guanosine monophosphate. For synthesis of GTP, shown in the upper right portion of the diagram, two more phosphate moieties are added stepwise to create guanosine diphosphate (GDP) first and then GTP. For synthesis of dGTP, a building block for DNA replication, GDP is converted to deoxyguanosine diphosphate (dGDP), which is then phosphorylated to produce dGTP.

Purines can also be synthesized from the components of degraded macromolecules via salvage pathways. The lower half of the diagram shows that IMP and GMP can be broken down to hypoxanthine and guanine, respectively, via multiple steps. However, hypoxanthine-guanine phosphoribosyltransferase (HGPRT) reverses both series of reactions to regenerate IMP and GMP under appropriate conditions. Thus, HGPRT bypasses IMPDH to synthesize guanine nucleotides via the guanine salvage pathway. Certain tissues and organs are unable to undergo de novo synthesis of purines and rely exclusively on salvage pathways to supply needed nucleotides.

Hypoxanthine can be sequentially converted by xanthine oxidase (XO) first to xanthine and then to uric acid. Inhibition of IMPDH can therefore lead to a high concentration of uric acid in the blood, which may cause medical problems, including kidney stones, gout, and diabetes. Consequently, the therapeutic use of IMPDH inhibitors is usually accompanied by administration of an inhibitor of XO to prevent accumulation of uric acid in the body. Also, hypoxanthine inhibits HGPRT, so the administration of XO increases levels of hypoxanthine to potentiate this blockade of guanine salvage.

Humans have two IMPDH isozymes that have similar enzymatic characteristics. The differential roles of the two isozymes are not well understood.

Given the role of IMPDH in synthesis of guanine nucleotides, the invention recognizes that the metabolites in guanine nucleotide synthesis pathways are useful indicators for engagement of IMPDH inhibitors with their target. For example, inhibition of IMPDH prevents de novo synthesis of GTP, so decreased GTP levels are indicative of the degree of IMPDH inhibition in cells capable of de novo GTP synthesis. In addition, IMPDH inhibition results in the accumulation of the substrate IMP, which is then converted to hypoxanthine. As indicated above, conversion of hypoxanthine to uric acid can be blocked by providing inhibitors of XO. Therefore, in subjects that have received inhibitors of both IMPDH and XO, increased levels hypoxanthine are indicative of the degree of inhibition of IMPDH salvage.

### Compositions Containing IMPDH Inhibitors

Any IMPDH inhibitor known in the art may be used in embodiments of the invention. IMPDH inhibitors may be provided as active compounds, prodrugs, analogs, derivatives, micellar formulations, sustained release formulations, or salts. IMPDH inhibitors are described in, for example, Cuny, G. D., et al., Inosine-5′-monophosphate dehydrogenase (IMPDH) inhibitors: a patent and scientific literature review (2002-2016), Expert Opin Ther Pat., 2017, June; 27(6):677-690. doi: 10.1080/13543776.2017.1280463, the contents of which are incorporated herein by reference.

In some embodiments, the IMPDH inhibitor is tiazofurin. Tiazofurin, which has the systematic name 2-β-D-ribofuranosylthiazole-4-carboxamide, has the following structure:

Tiazofurin and methods for making it are known in the art and described in, for example, U.S. Pat. Nos. 4,451,648 and 6,613,896, the contents of which are incorporated herein by reference. Tiazofurin analogs, their activity against tumors, and methods of making them are described in, for example, Popsavin, et al., Synthesis and antiproliferative activity of two new tiazofurin analogues with 2′-amido functionalities, Bioorg. Med. Chem. Lett. 16 (2006) 2773-2776, doi: 10.1016/j.bmcl.2006.02.001; and Popsavin, et al., Synthesis and in vitro antitumor activity of tiazofurin analogues with nitrogen functionalities at the C-2′ position, European Journal of Medicinal Chemistry 111 (2016) 114e125. doi: 10.1016/j.ejmech.2016.01.037, the contents of each of which are incorporated herein by reference.

Tiazofurin is converted inside cells to thiazole-4-carboxamide adenine dinucleotide (TAD). TAD, an analog of NAD⁺, is the form of the compound that interacts with IMPDH to block its activity.

Another IMPDH inhibitor is the non-reversible inhibitor mycophenolic acid. Mycophenolic acid has the following structure:

Uses, side effects, and the mechanism of action of mycophenolate are known in the art and described in, for example, Kitchin, J. E., et al., (1997) “Rediscovering mycophenolic acid: A review of its mechanism, side effects, and potential uses” Journal of the American Academy of Dermatology. 37 (3): 445-449. doi:10.1016/S0190-9622(97)70147-6; and Pharmacology North American Edition. Lippincott Williams & Wilkins. 2014, p. 625, ISBN 978-1-4511-9177-6, the contents of each of which are incorporated herein by reference. Salts, derivatives, analogs, and prodrugs of mycophenolic acid are known in the art and described in, for example, Mele T. S., and Halloran, P. F., The use of mycophenolate mofetil in transplant recipients. Immunopharmacology. 2000; 47:215-245; International Publication No. WO 2004/096287A2; and U.S. Pat. No. 7,427,636, the contents of each of which are incorporated herein by reference.

Many other inhibitors of IMPDH are known in the art. For example and without limitation, other IMPDH inhibitors include AS2643361, EICAR, FF-10501, mizoribine, ribavirin, selenazofurin, SM-108, taribavirin, VX-148, VX-497, and VX-944. For example and without limitation, other IMPDH inhibitors include ribavirin, mizoribine, selenazofurin, and taribavirin. Other IMPDH inhibitors are described in Gebeyehu, G., et al., Ribavirin, Tiazofurin, and Selenazofurin: Mononucleotides and Nicotinamide Adenine Dinucleotide Analogues. Synthesis, Structure, and Interactions with IMP Dehydrogenase, J. Med. Chem. 1985, 28, 99-105; and U.S. Pat. Nos. 5,807,876; 6,344,465; 6,395,763; 6,399,773; 6,420,403; 6,518,291; 6,541,496; 6,617,323; 6,624,184; 6,653,309; 6,825,224; 6,867,299; 6,919,335; 6,967,214; 7,053,111; 7,060,720; 7,087,642; 7,205,324; 7,329,681; 7,432,290; 7,777,069; and 7,989,498, the contents of each of which are incorporated herein by reference.

As described above, it is desirable to administer an IMPDH inhibitor in conjunction with an inhibitor of xanthine oxidase to prevent accumulation of uric acid. Several xanthine oxidase inhibitors are known in the art. For example and without limitation, the inhibitor of xanthine oxidase may be allopurinol, oxypurinol, tisopurine, topiroxostat, phytic acid, or myoinositol.

The IMPDH inhibitors, including prodrugs, analogs, derivatives, and salts thereof, may be provided as pharmaceutical compositions. Pharmaceutical compositions containing the IMPDH inhibitors described above are known in the art and may be suitable for delivery by any suitable mechanism. Exemplary compositions are described below.

Except as otherwise indicated above, a pharmaceutical composition may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents, and preserving agents in order to provide pharmaceutically elegant and palatable preparations. Tablets contain the compounds in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example starch, gelatin, or acacia; and lubricating agents, for example magnesium stearate, stearic acid, or talc.

Oral compositions, such as tablets, capsules, liquids, etc., may be formulated for rapid release or sustained release. Solid formulations, such as tablets or capsules, may be uncoated, or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release. Preparation and administration of compounds is discussed in U.S. Pat. No. 6,214,841 and U.S. Pub. No. 2003/0232877, the contents of each of which are incorporated by reference herein.

Formulations for oral use may also be presented as hard gelatin capsules in which the compounds are mixed with an inert solid diluent, for example calcium carbonate, calcium phosphate, or kaolin, or as soft gelatin capsules in which the compounds are mixed with water or an oil medium, for example peanut oil, liquid paraffin, or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth, and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin, or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables.

Pharmaceutical compositions may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin (glycerol), erythritol, xylitol. sorbitol, mannitol, and polyethylene glycol; esters, such asethyl oleate and ethyllaurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. The pharmaceutically acceptable carrier may be an encapsulation coating. For example, the encapsulation coating may contain carrageenan, cellulose acetate phthalate, cellulose acetate succinate, cellulose acetate trimellitate, collagen, gelatin, hydroxypropyl methyl cellulose acetate, a methyl acrylate-methacrylic acid copolymer, polyvinyl acetate phthalate shellac, sodium alginate, starch, or zein.

The agents, such as IMPDH inhibitors or xanthine oxidase inhibitors, may be provided as pharmaceutically acceptable salts, such as nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphor sulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Other pharmaceutically acceptable salts may be found in, for example, Remington, The Science and Practice of Pharmacy (20th ed. 2000). Representative alkali or alkaline earth metal salts include sodium, lithium, potassium, calcium, magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

### Measuring the Level of a Metabolite in a Sample

Methods of the invention may include one or more of receiving information regarding a measured level of a metabolite in a sample, analyzing a measured level of a metabolite in a sample, and measuring a level of a metabolite in a sample. The metabolite may be GTP, IMP, XMP, or hypoxanthine.

In some embodiments, the metabolite is measured by mass spectrometry, optionally in combination with liquid chromatography. Molecules may be ionized for mass spectrometry by any method known in the art, such as ambient ionization, chemical ionization (CI), desorption electrospray ionization (DESI), electron impact (EI), electrospray ionization (ESI), fast-atom bombardment (FAB), field ionization, laser ionization (LIMS), matrix-assisted laser desorption ionization (MALDI), paper spray ionization, plasma and glow discharge, plasma-desorption ionization (PD), resonance ionization (RIMS), secondary ionization (SIMS), spark source, or thermal ionization (TIMS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference.

In certain embodiments, a sample can be directly ionized without the need for use of a separation system. In other embodiments, mass spectrometry is performed in conjunction with a method for resolving and identifying ionic species. Suitable methods include chromatography, capillary electrophoresis-mass spectrometry, and ion mobility. Chromatographic methods include gas chromatography, liquid chromatography (LC), high-pressure liquid chromatography (HPLC), hydrophilic interaction chromatography (HILIC), ultra-performance liquid chromatography (UPLC), and reversed-phase liquid chromatography (RPLC). In a preferred embodiment, liquid chromatography-mass spectrometry (LC-MS) is used. Methods of coupling chromatography and mass spectrometry are known in the art and described in, for example, Holcapek and Brydwell, eds. Handbook of Advanced Chromatography/Mass Spectrometry Techniques, Academic Press and AOCS Press (2017), ISBN 9780128117323; Pitt, Principles and Applications of Liquid Chromatography-Mass Spectrometry in Clinical Biochemistry, The Clinical Biochemist Reviews. 30(1): 19-34 (2017) ISSN 0159-8090; Niessen, Liquid Chromatography-Mass Spectrometry, Third Edition. Boca Raton: CRC Taylor & Francis. pp. 50-90. (2006) ISBN 9780824740825; Ohnesorge et al., Quantitation in capillary electrophoresis-mass spectrometry, Electrophoresis. 26 (21): 3973-87 (2005) doi:10.1002/elps.200500398; Kolch, et al., Capillary electrophoresis-mass spectrometry as a powerful tool in clinical diagnosis and biomarker discovery, Mass Spectrom Rev. 24 (6): 959-77. (2005) doi:10.1002/mas.20051; Kanu et al., Ion mobility-mass spectrometry, Journal of Mass Spectrometry, 43 (1): 1-22 (2008) doi:10.1002/jms.1383, the contents of which are incorporated herein by reference.

A sample may be obtained from any organ or tissue in the individual to be tested, provided that the sample is obtained in a liquid form or can be pre-treated to take a liquid form. For example and without limitation, the sample may be a blood sample, a urine sample, a serum sample, a semen sample, a sputum sample, a lymphatic fluid sample, a cerebrospinal fluid sample, a plasma sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, a phlegm sample, a saliva sample, a sweat sample, or a combination of such samples. A cell sample may be enriched for a specific type, class, or category of cells. For example, the cell sample may be a white blood cell sample. The sample may also be a solid or semi-solid sample, such as a tissue sample, feces sample, or stool sample, that has been treated to take a liquid form by, for example, homogenization, sonication, pipette trituration, cell lysis, etc. For the methods described herein, it is preferred that a sample is from plasma, serum, whole blood, or sputum.

The sample may be kept in a temperature-controlled environment to preserve the stability of the metabolite. For example, some compounds are more stable at lower temperatures, and the increased stability facilitates analysis of this metabolite from samples. Thus, samples may be stored at 4° C., −20° C., or −80° C. Alternatively or additionally, the sample may be maintained at ambient temperatures.

In some embodiments, a sample is treated to remove cells or other biological particulates. Methods for removing cells from a blood or other sample are well known in the art and may include e.g., centrifugation, sedimentation, ultrafiltration, immune selection, etc.

The subject may be an animal. For example, the subject may be a mammal, such as a human. The subject may be a pediatric, a newborn, a neonate, an infant, a child, an adolescent, a pre-teen, a teenager, an adult, or an elderly subject. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

The sample may be obtained from an individual before or after administration to the subject of an agent that alters activity of a metabolic pathway, such as inhibitor of an enzyme in the pathway. For example, the sample may be obtained 1 hour, 2 hours, 4 hours, 6 hours, 8 hours, 12 hours, 24 hours, 36 hours, 2 days, 3 days, 4 days, 5 days, 6 days, 7 days, or more before administration of an agent, or it may be obtained 1 hour, 2 hours, 4 hours, 6 hours, 8 hours, 12 hours, 24 hours, 36 hours, 2 days, 3 days, 4 days, 5 days, 6 days, 7 days, or more after administration of an agent.

Levels of a metabolite may be measured within a fraction or compartment of a sample. For example, metabolite levels may be intracellular metabolite levels or extracellular metabolite levels.

### Determining a Dosing Regimen

Methods of the invention include determining a dosing regimen of an IMPDH inhibitor for a subject. The dosing regimen may include a dose, i.e., an amount, of the IMPDH inhibitor that should be administered. The dosing regimen may include a time point for administration of a dose of the IMPDH inhibitor to the subject. Because the dosing regimen is based on one or more measured levels of a metabolite in a sample obtained from the subject, the dosing regimen is tailored to an individual subject, e.g., a patient. Consequently, the methods of the invention provide customized dosing regimens that account for variability in pharmacokinetic properties, i.e., metabolism of the active pharmaceutical ingredient (API) by the subject, and pharmacodynamics properties, i.e., effect of the API on its target, among individuals.

The dosing regimen may be determined by comparing a measured level of a metabolite in a sample obtained from a subject to a reference that provides an association between the measured level and a recommended dosage adjustment of the IMPDH inhibitor. For example, the reference may provide a relationship between administration of the IMPDH inhibitor and levels of the metabolite in the subject. The relationship can be empirically determined from a known dose and time of administration of the IMPDH inhibitor and measured levels of the metabolite at one or more subsequent time points. The reference may include a relationship between measured levels of the IMPDH inhibitor or a metabolic product of the IMPDH inhibitor and measured levels of the metabolite.

From the comparison between the measured level of a metabolite and the reference, a dosing regimen may then be determined. The dosing regimen may include a dosage of the IMPDH inhibitor, a time for administration of the dosage, or both. The dosing regimen may be determined de novo, or it may comprise an adjustment to a previous dosing regimen, such as an adjustment in the dosage, the interval between administration of dosages, or both.

The dosing regimen is designed to deliver the IMPDH inhibitor to the subject in an amount that achieves a therapeutic effect. The therapeutic effect may be a change in a sign or symptom of a disease, disorder, or condition. The therapeutic effect may be inhibition of an enzyme in the metabolic pathway, or it may be a change in an indicator of inhibition of an enzyme in a metabolic pathway. The indicator may be a metabolite in the pathway, and the therapeutic effect may be an increase or decrease in levels of a metabolite. The therapeutic effect may be a decrease in number of cancer cells, a decrease in proliferation of cancer cells, an increase in differentiation of pre-cancerous cells, such as myeloblasts, complete remission of cancer, complete remission with incomplete hematologic recovery, morphologic leukemia-free stat, or partial remission. Increased differentiation of myeloblasts may be assessed by one or more of expression of CD14, expression of CD11b, nuclear morphology, and cytoplasmic granules.

The dosing regimen may ensure that levels of a metabolite, such as hypoxanthine or XMP, are raised or maintained above a minimum threshold required to achieve a certain effect. For example, the dosing regimen may raise or maintain levels of a metabolite above a threshold level in the subject for a certain time period. The time period may include a minimum, a maximum, or both. For example, the dosing regimen may raise or maintain levels of a metabolite above the threshold level for at least 6 hours, at least 12 hours, at least 24 hours, at least 48 hours, at least 72 hours, at least 84 hours, at least 96 hours, at least 5 days, at least 6 days, at least 7 days, at least 10 days, at least 11 days, at least 12 days, at least 2 weeks, or more. The dosing regimen may raise or maintain levels of a metabolite above the threshold level for not more than 24 hours, not more than 36 hours, not more than 48 hours, not more than 60 hours, not more than 72 hours, not more than 84 hours, not more than 96 hours, not more than 5 days, not more than 6 days, not more than 7 days, not more than 8 days, not more than 9 days, not more than 10 days, not more than 12 days, or not more than 2 weeks. The dosing regimen may raise or maintain levels of a metabolite above the threshold level for at least 24 hours but not more than 96 hours, at least 36 hours but not more than 96 hours, at least 48 hours but not more than 96 hours, at least 60 hours but not more than 96 hours, at least 72 hours but not more than 96 hours, at least 72 hours but not more than 5 days, at least 72 hours but not more than 6 days, at least 72 hours but not more than 7 days, at least 96 hours but not more than 7 days, or at least 5 days but not more than 7 days.

The dosing regimen may ensure that levels of a metabolite, such as GTP or IMP, are lowered or maintained below a maximum threshold required to achieve a certain effect. For example, the dosing regimen may lower or maintain levels of a metabolite below a threshold level in the subject for a certain time period. The time period may include a minimum, a maximum, or both. For example, the dosing regimen may raise or maintain levels of a metabolite below the threshold level for at least 6 hours, 12, hours, 24 hours, at least 48 hours, at least 72 hours, at least 84 hours, at least 96 hours, at least 5 days, at least 6 days, at least 7 days, at least 8 days, at least 9 days, at least 10 days, at least 12 days, at least 2 weeks, or more. The dosing regimen may lower or maintain levels of a metabolite below the threshold level for not more than 24 hours, not more than 36 hours, not more than 48 hours, not more than 60 hours, not more than 72 hours, not more than 84 hours, not more than 96 hours, not more than 5 days, not more than 6 days, not more than 7 days, not more than 8 days, not more than 9 days, not more than 10 days, not more than 12 days, or not more than 2 weeks. The dosing regimen may lower or maintain levels of a metabolite below the threshold level for at least 24 hours but not more than 96 hours, at least 36 hours but not more than 96 hours, at least 48 hours but not more than 96 hours, at least 60 hours but not more than 96 hours, at least 72 hours but not more than 96 hours, at least 72 hours but not more than 5 days, at least 72 hours but not more than 6 days, at least 72 hours but not more than 7 days, at least 96 hours but not more than 7 days, or at least 5 days but not more than 7 days.

The dosing regimen may ensure that levels of a metabolite, such as hypoxanthine or XMP, do not fall below or are maintained above a minimum threshold that is associated with inhibition of uric acid that is further associated with toxicity. The dosing regimen may ensure that levels of a metabolite, such as GTP or IMP, do not exceed or are maintained below a maximum threshold that is associated with toxicity. Levels of a metabolite above a maximum threshold or below a minimum threshold may indicate that the IMPDH inhibitor is causing or is likely to cause an adverse event in the subject. For example and without limitation, adverse events include cardiotoxicity, coma, confusion, drowsiness, headaches, hypertension, infection, liver function abnormalities, myalgia, nausea, neurotoxicity, pleuropericarditis, rash, seizures, somnolence and vomiting.

The dosing regimen may include a time point for administration of one or more subsequent doses to raise or maintain levels of a metabolite above a threshold level for a certain time period. The dosing regimen may include a time point for administration of one or more subsequent doses to lower or maintain levels of a metabolite below a threshold level for a certain time period. The time point for administration of a subsequent dose may be relative to an earlier time point. For example, the time point for administration of a subsequent dose may be relative to a time point when a previous dose was administered or a time point when a sample was obtained from a subject.

The dosing regimen may include a schedule for administration of doses. For example, doses may be administered at regular intervals, such as every 24 hours, every 36 hours, every 48 hours, every 60 hours, every 72 hours, every 84 hours, every 96 hours, every 5 days, every 6 days, every week, every 2 weeks, every 3 weeks, or every 4 weeks. Alternatively, doses may be administered according to a schedule that does not require precisely regular intervals. For example, doses may be administered once per week, twice per week, three times per week, four times per week, once per month, twice per month, three times per month, four times per month, five times per month, or six times per month.

The metabolite may be GTP or IMP, and the dosing regimen may ensure that the IMPDH inhibitor is provided to lower or maintain the level of GTP or IMP below a threshold level. The threshold level may be expressed in absolute units, or it may be expressed relative to a reference value. The reference value may be the level of GTP or IMP in a subject or group of subjects that have not received the IMPDH inhibitor. The reference value may be the level of GTP or IMP in the same subject prior to having received the IMPDH inhibitor. The dosing regimen may ensure that the GTP level or IMP level remains less than about 20%, less than about 15%, less than about 10%, less than about 5%, from about 5% to less than about 20%, from about 10% to less than about 20%, from about 5% to about 15%, or from about 5% to about 10% of a reference level. The dosing regimen may ensure that the GTP level or IMP level remains at about 0%, about 5%, about 10%, about 15%, or about 20% of a reference level.

The metabolite may be hypoxanthine or XMP, and the dosing regimen may ensure that the IMPDH inhibitor is provided to raise or maintain the level of hypoxanthine or XMP above a threshold level. The threshold level may be expressed in absolute units, or it may be expressed relative to a reference value. The reference value may be the level of hypoxanthine or XMP in a subject or group of subjects that have not received the IMPDH inhibitor. The reference value may be the level of hypoxanthine or XMP in the same subject prior to having received the IMPDH inhibitor. The dosing regimen may ensure that the hypoxanthine level or XMP level remains at from about 250% to about 2500%, from about 400% to about 2000%, from about 500% to about 1500%, from about 600% to about 1400%, from about 700% to about 1300%, from about 800% to about 1200%, from about 900% to about 1100%, about 800%, about 900%, about 1000%, about 1100%, or about 1200% of a reference level. The dosing regimen may ensure that the hypoxanthine level or XMP level remains above about 400%, about 500%, about 600%, about 700%, about 800%, about 900%, about 1000%, about 1100%, or about 1200% of a reference level.

Any suitable level may be used as a reference level of a metabolite, such as GTP, IMP, XMP, or hypoxanthine. The reference level may be a level of the metabolite in a subject prior to administration of a composition to the subject or a level of the metabolite in a subject that has not received the composition. The reference level may be a level of the metabolite in the same subject prior to administration of the composition to the subject. Thus, the reference level may be the baseline metabolite level of the subject. The reference level of may be an average level of the metabolite in a group of subjects prior to administration of the composition to the subjects or an average level of the metabolite in subjects that have not received the composition. Thus, the reference level may be the average baseline metabolite level of a group of subjects.

Dosage of the IMPDH inhibitor also depends on factors such as the type of subject and route of administration. The dosage may fall within a range for a given type of subject and route of administration, or the dosage may be adjusted by a specified amount for a given type of subject and route of administration.

For example and without limitation, dosage of tiazofurin for intravenous or oral administration may be from about 200 mg/m² to about 12,000 mg/m², from about 500 mg/m² to about 10,000 mg/m², from about 1000 mg/m² to about 8000 mg/m², from about 2000 mg/m² to about 5000 mg/m², from about 3000 mg/m² to about 4000 mg/m², about 1000 mg/m², about 2000 mg/m², about 3000 mg/m², about 3300 mg/m², about 4000 mg/m², about 5000 mg/m², about 6000 mg/m², about 7000 mg/m², about 7600 mg/m², about 8000 mg/m², from about 0.8 g to about 12 g, from about 1 g to about 10 g, from about 2 g to about 6 g, from about 3 g to about 5 g, about 0.6 g, about 0.8 g, about 1 g, about 2 g, about 3 g, about 4 g, about 5 g, about 6 g, about 8 g, about 10 g, about 12 g, or about 15 g

For example and without limitation, tiazofurin may be administered intravenously daily for 2 days, 3 days, 4 days, 5 days, 6 days, 7 days, 8 days, 10 days, 12 days, 14 days or more. After a period of regular administration of tiazofurin for one or more days, there may be a drug-free period in which the subject does not receive tiazofurin. The drug-free period may be 1 day, 2 days, 3 days, 4 days, 5 days, 6 days, 7 days, 8 days, 10 days, 12 days, 2 weeks, 3 weeks, 4 weeks, 5 weeks, 6 weeks, 7 weeks, 8 weeks, or more.

For example and without limitation, tiazofurin may be administered by intravenous infusion for about 5 minutes, about 10 minutes, about 15 minutes, about 20 minutes, about 30 minutes, about 40 minutes, about 50 minutes, about 60 minutes, about 75 minutes, about 90 minutes, about 2 hours, about 2.5 hours, about 3 hours, about 4 hours, about 5 hours, about 6 hours, about 7 hours, or about 8 hours.

For example and without limitation, tiazofurin may be administered orally one time, two times, three times, four times, five times, six times, eight times, or more in a 24-hour period. For example and without limitation, tiazofurin may be administered orally once every 2 hours, 3 hours, 4 hours, 6 hours, 8 hours, 12 hours, or 24 hours.

### Evaluating Renal Function

Determining the dosing regimen for a subject may include evaluating renal function in the subject. Individuals with diminished renal function may display decreased rates of clearance of an IMPDH inhibitor from the body. Consequently, it may be necessary to decrease the dosage and/or frequency of administration of the IMPDH inhibitor to individuals with impaired renal function. In such instances, the degree of modification of a dosage regimen depends on the extent of impairment of renal function.

Any suitable metric may be used for evaluation of renal function. Two common measures of renal function are glomerular filtration rate (GFR) and creatinine clearance rate (C_{Cr}, CrCl, or CLcr).

GFR is the volume of fluid filtered from the renal glomerular capillaries into the Bowman's capsule per unit time. For a solute that is neither reabsorbed nor secreted by the kidneys, GFR is equal to the clearance rate and is represented by the following formula: GFR=(urine concentration*urine flow)/plasma concentration. Therefore, GFR can be determined by injection into a subject of a measurable substance that is not reabsorbed or secreted by the kidneys. For example and without limitation, suitable substances includes inulin, sinistrin, ⁵¹Cr-EDTA, and ^{99m}Tc-DTPA (pentetic acid). Use of such compounds to determine GFR is described in detail in Rose G A (1969), Measurement of glomerular filtration rate by inulin clearance without urine collection, BMJ, 2:91-3, doi:10.1136/bmj.2.5649.91; Murray, A. W. et al., Assessment of Glomerular Filtration Rate Measurement with Plasma Sampling: A Technical Review, Journal of Nuclear Medicine Technology, 41(2):67-75, doi:10.2967/jnmt.113.121004; Speeckaert, Marijn; Delanghe, Joris (2015), Assessment of renal function, In Giuseppe, Daniel; Winearls, Christopher, and Remuzzi, Giuseppe, Oxford Textbook of Clinical Nephrology (Fourth ed.), Oxford: Oxford University Press, p. 44. ISBN 9780199592548; Henriksen, Ulrik L.; Henriksen, Jens H. (January 2015), The clearance concept with special reference to determination of glomerular filtration rate in patients with fluid retention, Clinical Physiology and Functional Imaging, 35(1):7-16, doi:10.1111/cpf.12149; Soveri, Inga; Berg, et al., (September 2014), Measuring GFR: A Systematic Review, American Journal of Kidney Diseases. 64(3):411-424. doi:10.1053/j.ajkd.2014.04.010; Hsu, C.-y. and Bansal, N. (22 Jul. 2011), Measured GFR as Gold Standard—All that Glitters Is Not Gold?, Clinical Journal of the American Society of Nephrology, 6(8):1813-1814, doi:10.2215/CJN.06040611, PMID 21784836, the contents of each of which are incorporated herein by reference.

Creatinine clearance rate is the volume of blood plasma cleared of creatinine per unit time. Creatinine, which is produced from the breakdown of creatine phosphate in muscles, is freely filtered by the glomerulus and secreted only in small amounts. Consequently, C_{Cr} is a reasonable approximation of GFR. If the amount of secreted creatinine is ignored, GFR can be obtained from creatinine according to the following equation:

GFR=(urine creatinine concentration*urine volume)/serum creatinine concentration.

Measurement of C_{Cr} relies on a natural metabolite that is present in the blood in steady-state concentrations and does not require introduction of foreign substances into the body. Therefore, it is used more commonly in clinical settings to evaluate renal function.

Several formulas have been devised to estimate GFR and/or C_{Cr}, and one or more may be used to evaluate renal function in embodiments of the invention. One such formula is the Cockcroft-Gault formula:

estimated C_{Cr}=(140−age)*mass*constant/serum creatinine,

in which mass is the subject's mass in kg, serum creatinine is provided μmon, and the constant is 1.23 for men and 1.04 for women. Other suitable formulas include the Modification of Diet in Renal Disease (MDRD) formula, Chronic Kidney Disease Epidemiology Collaboration (CKD-EPI) formula, Mayo Quadratic formula, and Schwartz formula, which are known in the art and described in, for example, Levey A S, et al., (March 1999), A more accurate method to estimate glomerular filtration rate from serum creatinine: a new prediction equation. Modification of Diet in Renal Disease Study Group, Annals of Internal Medicine, 130(6):461-70, doi:10.7326/0003-4819-130-6-199903160-00002, PMID 10075613; Mathew T H, et al., (October 2007), Chronic kidney disease and automatic reporting of estimated glomerular filtration rate: revised recommendations, The Medical Journal of Australia, 187(8):459-63, PMID 17937643; Levey A S, et al., (May 2009), A new equation to estimate glomerular filtration rate, Annals of Internal Medicine, 150(9):604-12, doi:10.7326/0003-4819-150-9-200905050-00006, PMC 2763564, PMID 19414839; Rule A D, et al., (December 2004), Using serum creatinine to estimate glomerular filtration rate: accuracy in good health and in chronic kidney disease, Annals of Internal Medicine, 141(12):929-37, doi:10.7326/0003-4819-141-12-200412210-00009. PMID 15611490; Schwartz G J, et al., (August 1976), A simple estimate of glomerular filtration rate in children derived from body length and plasma creatinine, Pediatrics, 58(2):259-63, PMID 951142; Schwartz G J, et al, (June 1984), A simple estimate of glomerular filtration rate in full-term infants during the first year of life, The Journal of Pediatrics, 104(6):849-54, doi:10.1016/S0022-3476(84)80479-5, PMID 6726515, the contents of each of which are incorporated herein by reference.

Urea is another useful for marker for evaluating renal function. Blood urea nitrogen testing involves measurement of urea nitrogen in the blood. BUN levels are included in the MDRD formula.

Proteins, such as globulins and albumin, may also be used to evaluate renal function. Elevated levels of these and other proteins may be present in the urine, and proteinuria is an indicator of some kidney diseases. Albumin levels are also included in the MDRD formula.

Evaluating of renal function may include comparing a value for a parameter, such as GFR or C_{Cr}, to a reference value. The reference value may be a measured value. For example, the reference value may be a value from an individual or an average value from a group of individuals that have a common characteristic, such as normal renal or abnormal renal function. The reference value may be a predicted value for a subject. The predicted value may incorporate characteristics of the subject, such as the subject's age, weight, sex, ethnicity, genetic or genomic traits, diet, or other health conditions.

Determining the dosing regimen for a subject may involve determining a reference value of a parameter, such as GFR or C_{Cr}, of renal function for a subject and comparing the reference value to a measured value determined by one of the methods described above. The dosing regimen may be adjusted if the measured value is reduced compared to reference value. For example and without limitation, the dosing regimen may be adjusted if the measured value is less than about 90%, less than about 80%, less than about 70%, less than about 60%, less than about 50%, less than about 40%, less than about 30%, less than about 20%, or less than about 10% of a reference value.

Determining the dosing regimen may include determining that a subject has a measure value of a parameter, such as GFR or C_{Cr}, of renal function that meets a threshold compared to a reference value. For example and without limitation, the dosing regimen may be provided only if the measured value is at least about 10%, at least about 20%, at least about 30%, at least about 40%, at least about 50%, at least about 60%, at least about 70%, at least about 80%, or at least about 90% of the reference level.

Any element of the dosing regimen may be adjusted based on an evaluation of the subject's renal function. For example, the dosage, the frequency or administration, or both may be adjusted based on the evaluation of the subject's renal function. For example and without limitation, for a subject that has reduced renal function, the dosage may be decreased, the frequency of administration may be decreased, or both may be decreased.

Evaluation of renal function may be performed at any point during the methods of the invention. In methods that include a first phase in which a composition containing a IMPDH inhibitor is provided to a subject and a second phase in which the composition is not provided to the subject, renal function may be evaluated prior to the first phase, during the first phase, at the end of the first phase, at the start of the second phase, during the second phase, or at the end of the second phase, or at any combination of such time points. In methods in which the aforementioned first and second phase are repeated as cycles, renal function may be at any point during one or more cycles.

### Providing Doses of an IMPDH Inhibitor

Methods of the invention may include providing an IMPDH inhibitor to a subject according to a dosing regimen or dosage determined as described above. Providing the IMPDH inhibitor to the subject may include administering it to the subject. A dose may be administered as a single unit or in multiple units. The IMPDH inhibitor may be administered by any suitable means. For example and without limitation, the IMPDH inhibitor may be administered orally, intravenously, enterally, parenterally, dermally, topically, transdermally, by injection, intravenously, subcutaneously, nasally, pulmonarily, or with or on an implantable medical device (e.g., stent or drug-eluting stent or balloon equivalents). Oral administration of tiazofurin is described in Obeng, E. K., et al., Pharmacokinetics of tiazofurin in dogs, Biopharm Drug Dispos. 1987 March-April; 8(2):125-32, the contents of which are incorporated herein by reference.

Embodiments of methods of providing doses are described below with reference to measured levels of a metabolite and threshold levels of the metabolite. Any metabolite in a purine synthesis pathway or purine degradation pathway may be used. Preferably, the metabolite is GTP or hypoxanthine. As described above, it will be understood that threshold levels of GTP may be maximums, and measured levels of the metabolite below the threshold indicate inhibition of IMPDH. Conversely, threshold levels of hypoxanthine may be minimums, and measured levels of the metabolite below the threshold indicate inhibition of IMPDH salvage.

In some embodiments, the levels of multiple metabolites are measured, and any of the aforementioned metabolites may be used. In some embodiments, the metabolites measured are GTP and hypoxanthine.

In some embodiments, the methods include assessing a metabolite level in a sample from a subject, and determining whether that level is within a threshold range that warrants dosing, and/or that warrants dosing at a particular level or in a particular amount. The methods may include assessing the levels of multiple metabolites to determine whether dosing is warranted.

The methods may include administering at least one dose of the IMPDH inhibitor to a subject whose plasma metabolite level has been determined and is above or below a pre-determined threshold. In some embodiments, the predetermined threshold reflects percent inhibition of IMPDH in the subject relative to a reference determined for the subject. In some embodiments, the reference is determined by an assay.

For example, in some embodiments, in order to maintain inhibition of IMPDH at an effective threshold, multiple doses of the IMPDH inhibitor may be administered. In some embodiments, dosing of the IMPDH inhibitor can occur at different times and in different amounts. The present disclosure encompasses those methods that can maintain inhibition of IMPDH at a consistent level at or above the efficacy threshold throughout the course of treatment. In some embodiments, the amount of inhibition of IMPDH is measured by the amount of metabolite in the plasma of a subject.

In some embodiments, more than one dose of the IMPDH inhibitor is administered to the subject. In some embodiments, the method further comprises a step of re-determining the subject's plasma metabolite level after administration of the at least one dose. In some embodiments, the subject's plasma metabolite level is re-determined after each dose. In some embodiments, the method further comprises administering at least one further dose of the IMPDH inhibitor after the subject's plasma metabolite level has been determined again (e.g., after administering a first or previous dose), and is above or below the pre-determined threshold. If the subject's plasma metabolite level is determined not to be above or below a pre-determined threshold, dosing or escalation of dose levels can be discontinued. In some embodiments, therefore, no further dose of the IMPDH inhibitor is administered until the subject's plasma metabolite level has been determined to again be above or below a pre-determined threshold.

The methods may include administering an IMPDH inhibitor to a subject at a dosage level at or near a cell-lethal level. Such dosage can be supplemented with a later dose at a reduced level, or by discontinuing of dosing. For example, in some embodiments, the present disclosure provides a method of administering an IMPDH inhibitor to a subject in need thereof, the method comprising: administering a plurality of doses of an IMPDH inhibitor, according to a regimen characterized by at least first and second phases, wherein the first phase involves administration of at least one bolus dose of an IMPDH inhibitor at a cell-lethal level; and the second phase involves either: administration of at least one dose that is lower than the bolus dose; or absence of administration of an IMPDH inhibitor.

In some embodiments, an IMPDH inhibitor is not administered during a second phase. In some embodiments, a second phase involves administration of guanine rescue therapy. In some embodiments, a bolus dose is or comprises a cell lethal dose. In some embodiments, a cell lethal dose is an amount of an IMPDH inhibitor that is sufficient to cause apoptosis in normal (e.g., non-cancerous) cells in addition to target cells (e.g., cancer cells).

In some embodiments, the first phase and the second phase each comprise administering an IMPDH inhibitor. In some embodiments, the first phase and the second phase are at different times. In some embodiments, the first phase and the second phase are on different days. In some embodiments, the first phase lasts for a period of time that is less than four days. In some embodiments, the first phase comprises administering an IMPDH inhibitor, followed by a period of time in which no IMPDH inhibitor is administered. In some embodiments, the period of time in which no IMPDH inhibitor is administered is 3, 4, 5, 6, or 7 days after the providing the first dose in the first phase. In some embodiments, the first phase comprises administering more than one dose.

In some embodiments, an IMPDH inhibitor is administered during a second phase. In some embodiments, an IMPDH inhibitor is administered sub-cell-lethal levels during the second phase. In some embodiments, the first phase is repeated after the second phase. In some embodiments, both the first and second phases are repeated.

In some embodiments, the present disclosure provides a method of administering an IMPDH inhibitor to a subject in need thereof, according to a multi-phase protocol comprising: a first phase in which at least one dose of the IMPDH inhibitor is administered to the subject; and a second phase in which at least one dose of the IMPDH inhibitor is administered to the subject, wherein one or more doses administered in the second phase differs in amount and/or timing relative to other doses in its phase as compared with the dose(s) administered in the first phase.

In some embodiments, a metabolite level is determined in a sample from the subject between the first and second phases. In some embodiments, the sample is a plasma sample. In some embodiments, the timing or amount of at least one dose administered after the metabolite level is determined differs from that of at least one dose administered before the metabolite level was determined.

In some embodiments, the amount of IMPDH inhibitor that is administered to the patient is adjusted in view of the metabolite level in the subject's plasma. For example, in some embodiments, a first dose is administered in the first phase. In some embodiments, metabolite level is determined at a period of time after administration of the first dose.

In some embodiments, if the metabolite level is above or below a pre-determined level, the amount of IMPDH inhibitor administered in a second or subsequent dose is increased and/or the interval between doses is reduced. For example, in some such embodiments, the amount of IMPDH inhibitor administered may be increased. For example and without limitation, the amount of IMPDH inhibitor administered in a second or subsequent dose may be increased by about 100 mg/m², about 200 mg/m², about 400 mg/m², about 600 mg/m², about 800 mg/m², about 1 g/m², about 1.5 g/m², about 2 g/m², about 2.5 g/m², about 3 g/m², about 3.5 g/m², about 4 g/m², about 4.5 g/m², about 5 g/m², about 6 g/m², about 7 g/m², about 8 g/m², or about 10 g/m². In some embodiments, the amount of IMPDH inhibitor administered may be increased by an adjustment amount determined based on change in metabolite levels observed between prior doses of different amounts administered to the subject.

In some embodiments, if the metabolite level is above or below a pre-determined level, the amount of IMPDH inhibitor administered in a second or subsequent dose is the same as the amount administered in the first or previous dose and/or the interval between doses is the same.

In some embodiments, if the metabolite level is above or below a pre-determined level, the amount of IMPDH inhibitor in a second or subsequent dose is decreased and/or the interval between doses is increased. For example, in some such embodiments, the amount of IMPDH inhibitor administered may be decreased, for example, by 50 mg/m². In some embodiments, if the metabolite level is above or below a pre-determined level, the amount of IMPDH inhibitor in a second or subsequent dose is decreased by 75 mg/m². In some embodiments, if the metabolite level is above or below a pre-determined level, the amount of IMPDH inhibitor in a second or subsequent dose is decreased by 100 mg/m². In some embodiments, the amount of IMPDH inhibitor administered may be decreased by an adjustment amount determined based on change in metabolite levels observed between prior doses of different amounts administered to the subject.

In some embodiments, the present disclosure provides a method of administering a later dose of an IMPDH inhibitor to a patient who has previously received an earlier dose of the IMPDH inhibitor, wherein the patient has had a level of metabolite assessed subsequent to administration of the earlier dose, and wherein the later dose is different from the earlier dose. The later dose may be different from the earlier dose in amount of IMPDH inhibitor included in the dose, time interval relative to an immediately prior or immediately subsequent dose, or combinations thereof. The amount of IMPDH inhibitor in the later dose may be less than that in the earlier dose.

The method may include administering multiple doses of the IMPDH inhibitor, separated from one another by a time period that is longer than 2 days and shorter than 8 days for example, the time period may be about 3 days.

In some embodiments, the metabolite level is determined in a sample from the subject before each dose is administered, and dosing is delayed or skipped if the determined metabolite level is above or below a pre-determined threshold. For example, the metabolite level may be determined about 12 hours, about 24 hours, about 36 hours, about 48 hours, about 60 hours, about 72 hours, about 84 hours, or about 96 hours after administration of an IMPDH inhibitor

The method may include administering the IMPDH inhibitor according to a regimen approved in a trial in which a level of metabolite was measured in a patient between doses of the IMPDH inhibitor. The regimen may include multiple doses whose amount and timing were determined in the trial to maintain the metabolite level within a range determined to indicate a degree of IMPDH inhibition below a toxic threshold and above a minimum threshold. The regimen may include determining the metabolite level in the subject after administration of one or more doses of the IMPDH inhibitor.

In some embodiments, the regimen includes a dosing cycle in which an established pattern of doses is administered over a first period of time. In some embodiments, the regimen comprises a plurality of the dosing cycles. In some embodiments, the regimen includes a rest period during which the IMPDH inhibitor is not administered between the cycles.

### Conditions, Including Diseases and Disorders

The methods of the invention are useful for treating a variety of conditions, including cancer. The condition may be any disease or disorder for which inhibiting IMPDH is of therapeutic benefit.

The condition may be cancer. The cancer may include a solid tumor or hematological tumor. The cancer may be acute myeloid leukemia (AML), acute lymphoblastic leukemia (ALL), adult T cell leukemia/lymphoma (ATLL), bladder cancer, breast cancer, such as triple negative breast cancer (TNBC), glioma, head and neck cancer, leukemia, such as AML, lung cancer, such as small cell lung cancer and non-small cell lung cancer, lymphoma, multiple myeloma, neuroblastoma, osteosarcoma, ovarian cancer, prostate cancer, or renal cell cancer.

The condition may be an inflammatory or autoimmune disorder, such as arthritis, hepatitis, chronic obstructive pulmonary disease, multiple sclerosis, or tendonitis.

The condition may be a psychiatric disorder, such as anxiety, stress, obsessive-compulsive disorder, depression, panic disorder, psychosis, addiction, alcoholism, attention deficit hyperactivity, agoraphobia, schizophrenia, or social phobia.

The condition may be a neurological or pain disorder, such as epilepsy, stroke, insomnia, dyskinesia, peripheral neuropathic pain, chronic nociceptive pain, phantom pain, deafferentation pain, inflammatory pain, joint pain, wound pain, post-surgical pain, recurrent headache pain, an appetite disorder, or motor activity disorder.

The condition may be a neurodegenerative disorder, such as Alzheimer's disease, Parkinson's disease, or Huntington's disease.

The condition may be a metabolic disorder, such as diabetes, insulin resistance, or obesity.

The condition may be a skin condition, such as psoriasis.

The condition may be being the recipient of an organ transplant, such as a kidney transplant or liver transplant. The IMPDH inhibitor may be provided to suppress the recipient's immune system and prevent transplant rejection.

The condition may be an infection, such as a viral, bacterial, or fungal infection. The IMPDH inhibitor may be provided to suppress proliferation of an infectious microorganism. The microorganism may be a hepatitis virus, such as hepatitis C virus.

The condition may include a class or subset of patients having a disease, disorder, or condition. For example, AML cases are classified based on cytological, genetic, and other criteria, and AML treatment strategies vary depending on classification. One AML classification system is provided by the World Health Organization (WHO). The WHO classification system includes subtypes of AML provided in Table 1 and is described in Falini B, et al. (October 2010) “New classification of acute myeloid leukemia and precursor-related neoplasms: changes and unsolved issues” Discov Med. 10 (53): 281-92, PMID 21034669, the contents of which are incorporated herein by reference.

TABLE 1

| Name | Description |
|---|---|
| Acute myeloid | Includes: |
| leukemia with | AML with translocations between chromosome 8 and 21 - |
| recurrent | [t(8; 21)(q22; q22);] RUNX1/RUNX1T1; (ICD-O 9896/3); |
| genetic | AML with inversions in chromosome 16 - [inv(16)(p13.1q22)] or internal |
| abnormalities | translocations in it - [t(16; 16)(p13.1; q22);] CBFB/MYH11; (ICD-O 9871/3); |
| | Acute promyelocytic leukemia with translocations between chromosome |
| | 15 and 17 - [t(15; 17)(q22; q12);] RARA/PML; (ICD-O 9866/3); |
| | AML with translocations between chromosome 9 and 11- |
| | [t(9; 11)(p22; q23);] MLLT3/MLL; |
| | AML with translocations between chromosome 6 and 9 - |
| | [t(6; 9)(p23; q34);] DEK/NUP214; |
| | AML with inversions in chromosome 3 - [inv(3)(q21q26.2)] or internal |
| | translocations in it - [t(3; 3)(q21; q26.2);] RPN1/EVI1; |
| | Megakaryoblastic AML with translocations between chromosome |
| | 1 and 22 - [t(1; 22)(p13; q13);] RBM15/MKL1; |
| | AML with mutated NPM1 |
| | AML with mutated CEBPA |
| AML with | Includes people who have had a prior documented myelodysplastic |
| myelodysplasia- | syndrome (MDS) or myeloproliferative disease (MPD) that then has |
| related changes | transformed into AML, or who have cytogenetic abnormalities characteristic |
| | for this type of AML (with previous history of MDS or MPD that has gone |
| | unnoticed in the past, but the cytogenetics is still suggestive of MDS/MPD |
| | history). This category of AML occurs most often in elderly people and often |
| | has a worse prognosis. Includes: |
| | AML with complex karyotype |
| | Unbalanced abnormalities |
| | AML with deletions of chromosome 7 - [del(7q);] |
| | AML with deletions of chromosome 5 - [del(5q);] |
| | AML with unbalanced chromosomal aberrations in chromosome 17 - |
| | [i(17q)/t(17p);] |
| | AML with deletions of chromosome 13 - [del(13q);] |
| | AML with deletions of chromosome 11 - [del(11q);] |
| | AML with unbalanced chromosomal aberrations in chromosome 12 -[del(12p)/t(12p);] |
| | AML with deletions of chromosome 9 - [del(9q);] |
| | AML with aberrations in chromosome X - [idic(X)(q13);] |
| | Balanced abnormalities |
| | AML with translocations between chromosome 11 and 16 - |
| | [t(11; 16)(q23; q13.3);], unrelated to previous chemotherapy or ionizing radiation |
| | AML with translocations between chromosome 3 and 21 - |
| | [t(3; 21)(q26.2; q22.1);], unrelated to previous chemotherapy or |
| | ionizing radiation |
| | AML with translocations between chromosome 1 and 3 - |
| | [t(1; 3)(p36.3; q21.1);] |
| | AML with translocations between chromosome 2 and 11- |
| | [t(2; 11)(p21; q23);], unrelated to previous chemotherapy or ionizing |
| | radiation |
| | AML with translocations between chromosome 5 and 12 - |
| | [t(5; 12)(q33; p12);] |
| | AML with translocations between chromosome 5 and 7 - |
| | [t(5; 7)(q33; q11.2);] |
| | AML with translocations between chromosome 5 and 17 - |
| | [t(5; 17)(q33; p13);] |
| | AML with translocations between chromosome 5 and 10 - |
| | [t(5; 10)(q33; q21);] |
| | AML with translocations between chromosome 3 and 5 - |
| | [t(3; 5)(q25; q34);] |
| Therapy-related | Includes people who have had prior chemotherapy and/or radiation and |
| myeloid | subsequently develop AML or MDS. These leukemias may be characterized |
| neoplasms | by specific chromosomal abnormalities, and often carry a worse prognosis. |
| Myeloid | Includes myeloid sarcoma. |
| sarcoma | |
| Myeloid | Includes so-called “transient abnormal myelopoiesis” and “Myeloid leukemia |
| proliferations | associated with Down syndrome” |
| related to Down | |
| syndrome | |
| Blastic | Includes so-called “blastic plasmacytoid dendritic cell neoplasm” |
| plasmacytoid | |
| dendritic cell | |
| neoplasm | |
| AML not | Includes subtypes of AML that do not fall into the above categories |
| otherwise | AML with minimal differentiation |
| categorized | AML without maturation |
| | AML with maturation |
| | Acute myelomonocytic leukemia |
| | Acute monoblastic and monocytic leukemia |
| | Acute erythroid leukemia |
| | Acute megakaryoblastic leukemia |
| | Acute basophilic leukemia |
| | Acute panmyelosis with myelofibrosis |

An alternative classification scheme for AML is the French-American-British (FAB) classification system. The FAB classification system includes the subtypes of AML provided in Table 2 and is described in Bennett J M, et al. (August 1976), “Proposals for the classification of the acute leukemias. French-American-British (FAB) co-operative group” Br. J. Haematol. 33 (4): 451-8, doi:10.1111/j.1365-2141.1976.tb03563.x, PMID 188440; and Bennett J M, et al. (June 1989) “Proposals for the classification of chronic (mature) B and T lymphoid leukemias. French-American-British (FAB) Cooperative Group” J. Clin. Pathol. 42 (6): 567-84, doi:10.1136/jcp.42.6.567, PMC 1141984, PMID 2738163, the contents of each of which are incorporated herein by reference.

TABLE 2

| Type | Name | Cytogenetics |
|---|---|---|
| M0 | acute myeloblastic leukemia, minimally | |
| | differentiated | |
| M1 | acute myeloblastic leukemia, without maturation | |
| M2 | acute myeloblastic leukemia, with granulocytic | t(8; 21)(q22; q22), t(6; 9) |
| | maturation | |
| M3 | promyelocytic, or acute promyelocytic | t(15; 17) |
| | leukemia (APL) | |
| M4 | acute myelomonocytic leukemia | inv(16)(p13q22), del(16q) |
| M4eo | myelomonocytic together with bone | inv(16), t(16; 16) |
| | marrow eosinophilia | |
| M5 | acute monoblastic leukemia (M5a) or acute | del(11q), t(9; 11), t(11; 19) |
| | monocytic leukemia (M5b) | |
| M6 | acute erythroid leukemias, including | |
| | erythroleukemia (M6a) and very rare pure | |
| | erythroid leukemia (M6b) | |
| M7 | acute megakaryoblastic leukemia | t(1; 22) |

The methods may be used to treat other sub-populations of patients. For example, the patients may be pediatric, newborn, neonates, infants, children, adolescent, pre-teens, teenagers, adults, or elderly. The patients may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

### EXAMPLES

The following examples illustrate methods of providing an IMPDH inhibitor according to embodiments of the invention.

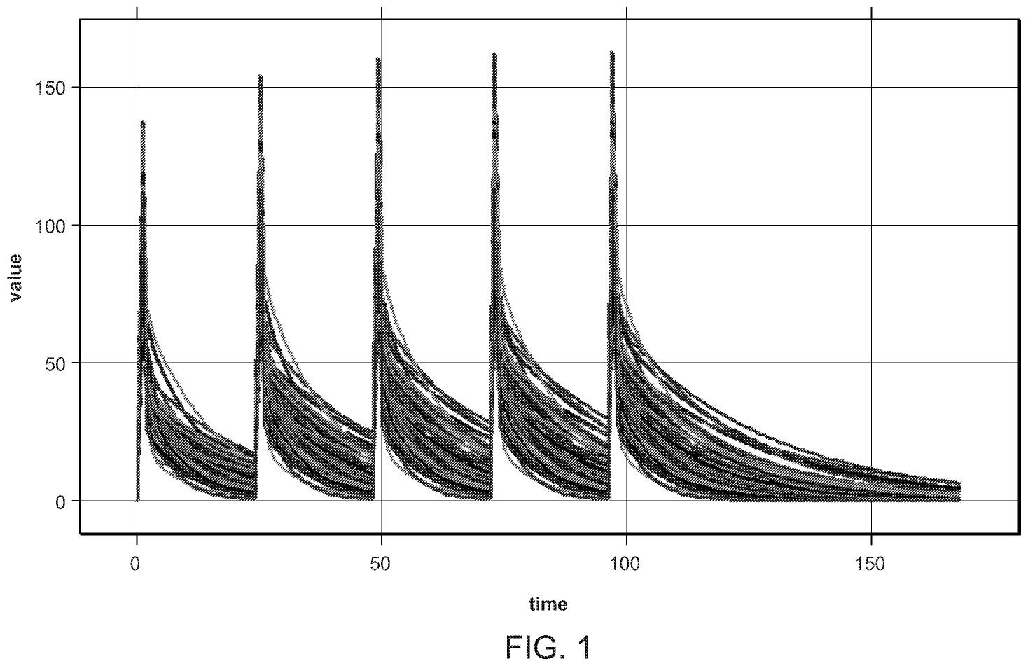

FIG. 1 is a graph of plasma tiazofurin levels in subjects after intravenous administration. Subjects are given 3800 mg tiazofurin intravenously every 24 hours for five days.

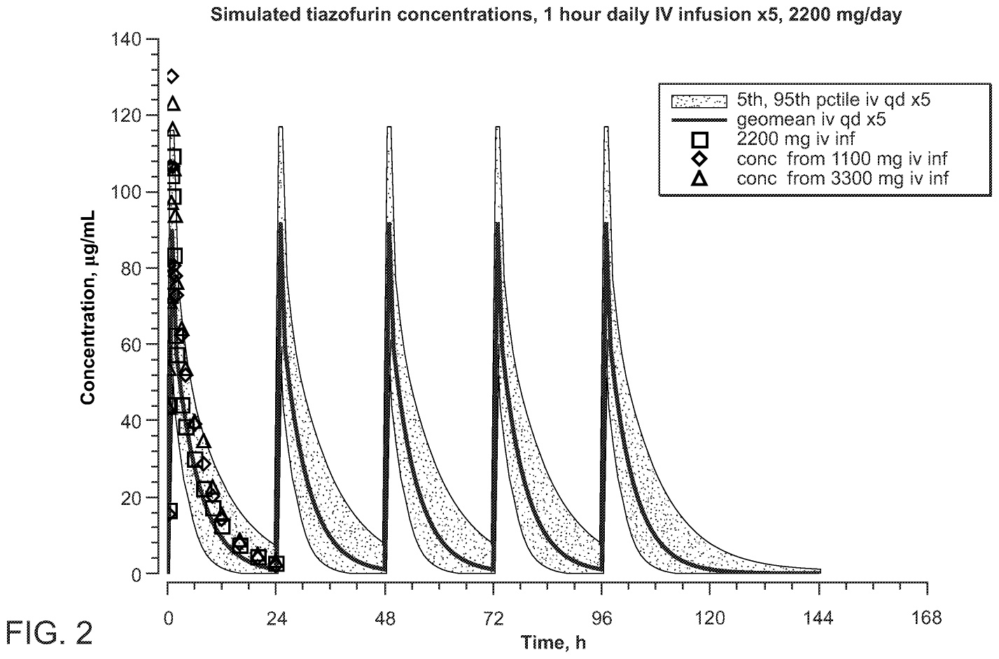

FIG. 2 is a graph of plasma tiazofurin levels in subjects after intravenous administration. A theoretical model of tiazofurin pharmacokinetics was developed based upon published human pharmacokinetic data for intravenous infusion of tiazofurin. From this model a simulation of daily, 1-hour intravenous infusions of 2,200 mg of tiazofurin for a period of 5 days was constructed. The model geometric mean with 5-95 percentiles are shown. To validate the model, the results from clinical pharmacokinetic data for doses of 1,100 mg and 3,300 mg intravenous 1-hour infusions were plotted and match the model very well. Dotted areas indicate values ranging from the 5th to 95th percentile; and solid lines indicate geometric mean values. Squares are values from subjects given a single, one-hour infusion of 2200 mg; diamonds are values from subjects given a single, one-hour infusion of 1100 mg; and triangles are values from subjects given a single, one-hour infusion of 3300 mg.

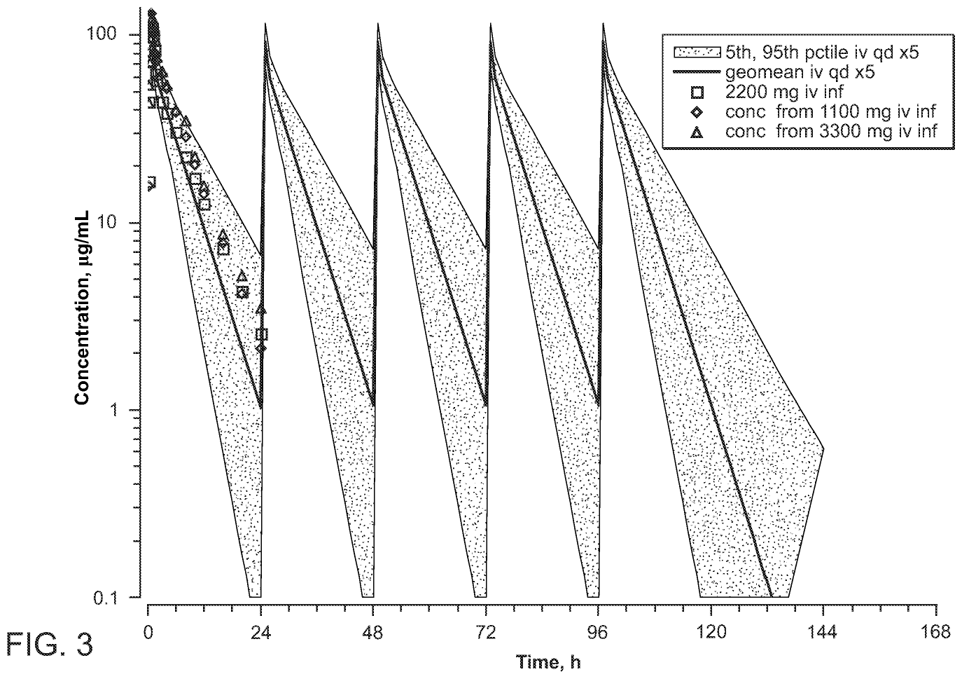

FIG. 3 is a graph of plasma tiazofurin levels in subjects after intravenous administration. The simulation data obtained as described above in relation to FIG. 2 was graphed using a logarithmic scale for the Y-axis. Dotted areas indicate values ranging from the 5th to 95th percentile; and black lines indicate geometric mean values. Squares are values from subjects given a single, one-hour infusion of 2200 mg; diamonds are values from subjects given a single, one-hour infusion of 1100 mg; and triangles are values from subjects given a single, one-hour infusion of 3300 mg.

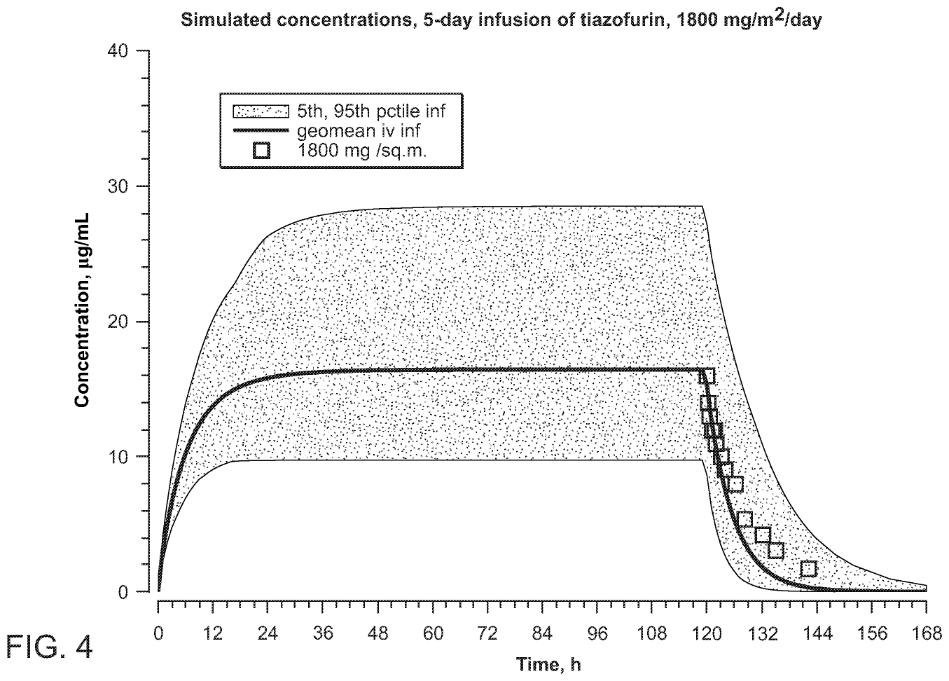

FIG. 4 is a graph of plasma tiazofurin levels in subjects after intravenous administration. Subjects are given 1800 mg/m² tiazofurin intravenously per day via continuous infusion for five days. Dotted areas indicate values ranging from the 5th to 95th percentile; and solid lines indicate geometric mean values. Squares are values from subject following the 1800 mg/m² dose.

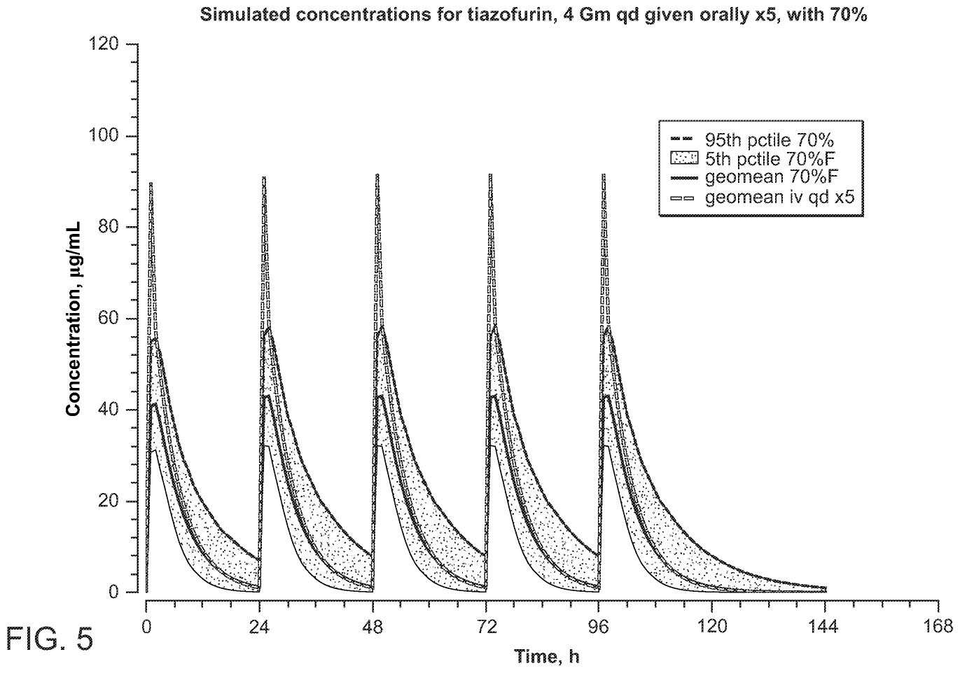

FIG. 5 is a graph of plasma tiazofurin levels in subjects after oral administration of a composition having 70% bioavailability. Subjects are given 4 g tiazofurin orally once every 24 hours for five days. Dotted areas indicate values ranging from the 5th to 95th percentile; solid lines indicate geometric mean values; and dashed lines indicate geometric mean values from control subjects given 4 g intravenously once every 24 hours.

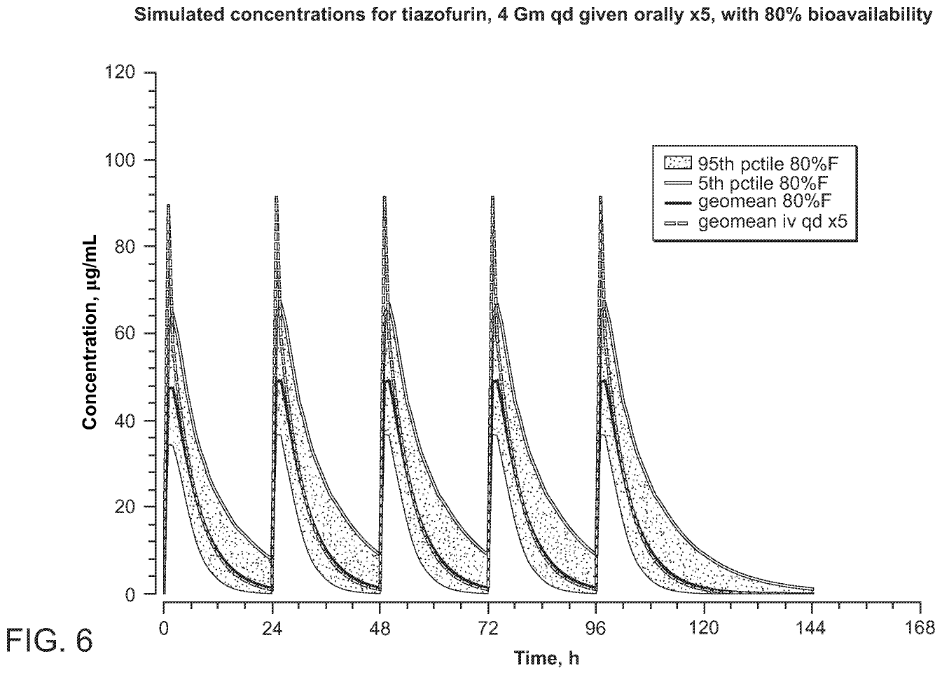

FIG. 6 is a graph of plasma tiazofurin levels in subjects after oral administration of a composition having 80% bioavailability. Subjects are given 4 g tiazofurin orally once every 24 hours for five days. Dotted areas indicate values ranging from the 5th to 95th percentile; solid lines indicate geometric mean values; and dashed lines indicate geometric mean values from control subjects given 4 g intravenously once every 24 hours.

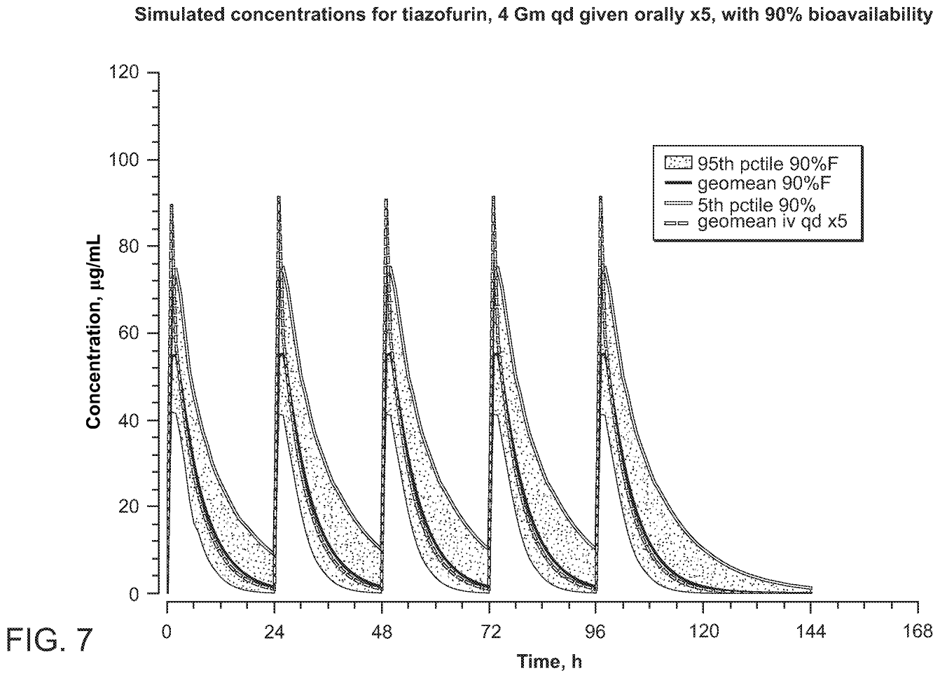

FIG. 7 is a graph of plasma tiazofurin levels in subjects after oral administration of a composition having 90% bioavailability. Subjects are given 4 g tiazofurin orally once every 24 hours for five days. Dotted areas indicate values ranging from the 5th to 95th percentile; solid lines indicate geometric mean values; and dashed lines indicate geometric mean values from control subjects given 4 g intravenously once every 24 hours.

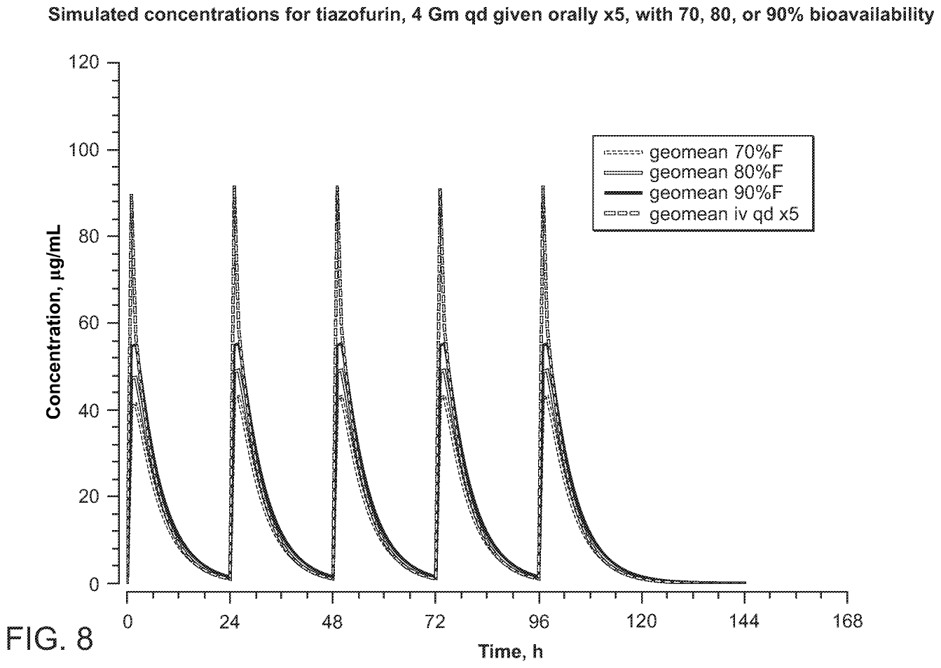

FIG. 8 is a graph of plasma tiazofurin levels in subjects after oral administration of a composition having 70%, 80%, or 90% bioavailability. Subjects are given 4 g tiazofurin orally once every 24 hours for five days. Continuous lines with dashed borders indicate geometric mean values following administration of a composition having 70%, bioavailability; continuous open lines indicate geometric mean values following administration of a composition having 80%, bioavailability; solid lines indicate geometric mean values following administration of a composition having 90%, bioavailability; and dashed open lines indicate geometric mean values from control subjects given 4 g intravenously once every 24 hours.

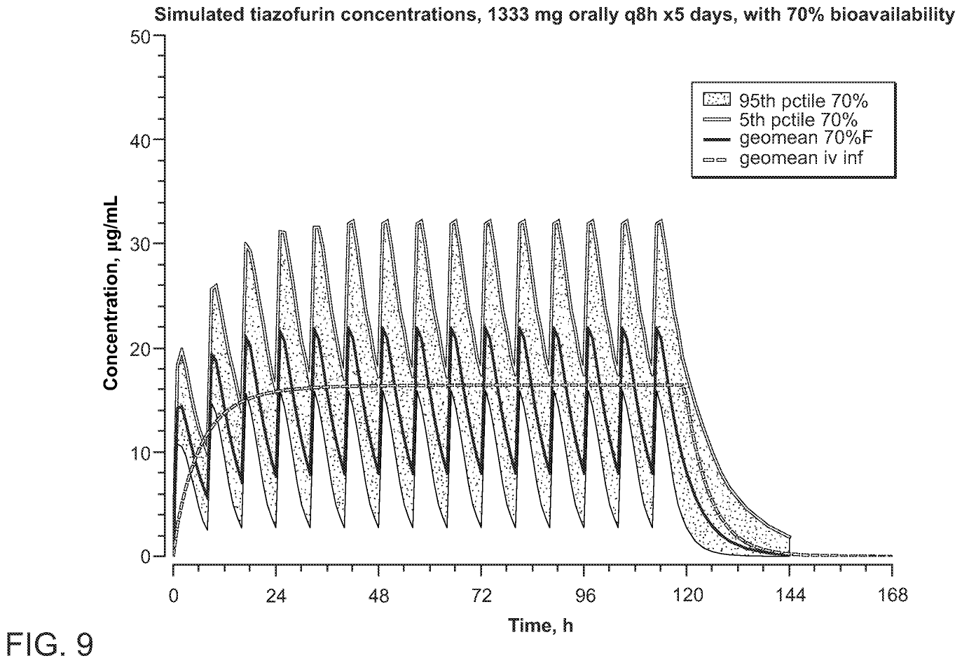

FIG. 9 is a graph of plasma tiazofurin levels in subjects after oral administration of a composition having 70% bioavailability. Subjects are given 1333 mg tiazofurin orally once every 8 hours for five days. Dotted areas indicate values ranging from the 5th to 95th percentile; solid lines indicate geometric mean values; and dashed lines indicate geometric mean values from control subjects given 4 g per day intravenously via continuous infusion for five days.

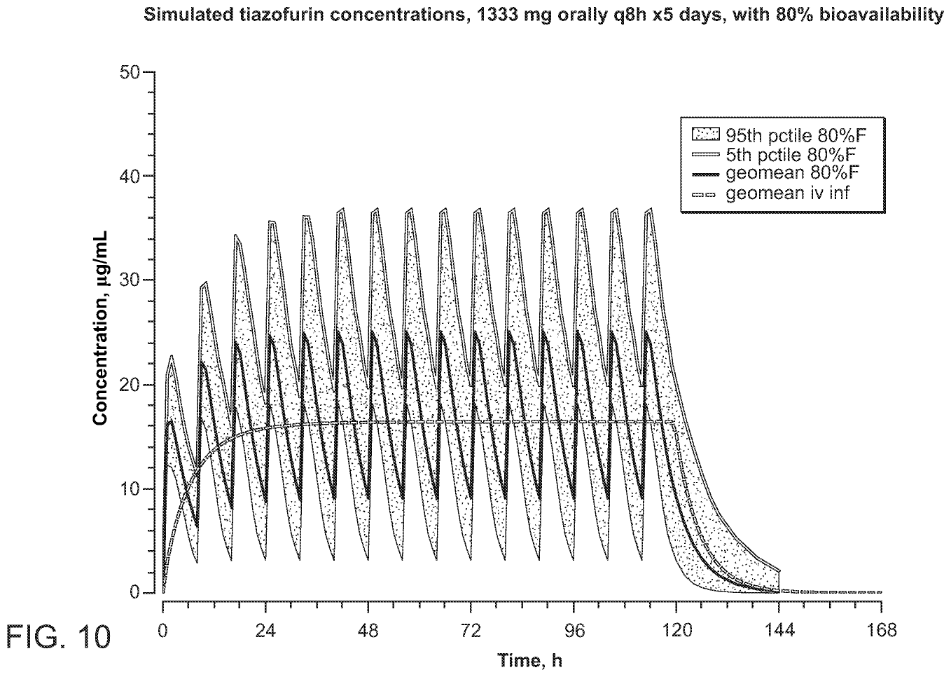

FIG. 10 is a graph of plasma tiazofurin levels in subjects after oral administration of a composition having 80% bioavailability. Subjects are given 1333 mg tiazofurin orally once every 8 hours for five days. Dotted areas indicate values ranging from the 5th to 95th percentile; solid lines indicate geometric mean values; and dashed open lines indicate geometric mean values from control subjects given 4 g per day intravenously via continuous infusion for five days.

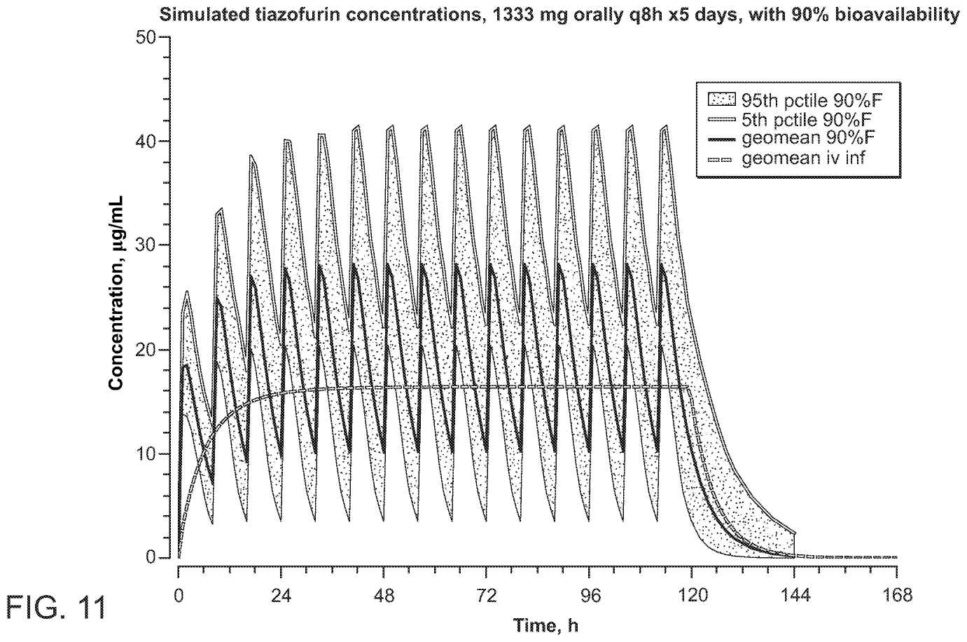

FIG. 11 is a graph of plasma tiazofurin levels in subjects after oral administration of a composition having 90% bioavailability. Subjects are given 1333 mg tiazofurin orally once every 8 hours for five days. Dotted areas indicate values ranging from the 5th to 95th percentile; solid lines indicate geometric mean values; and dashed lines indicate geometric mean values from control subjects given 4 g per day intravenously via continuous infusion for five days.

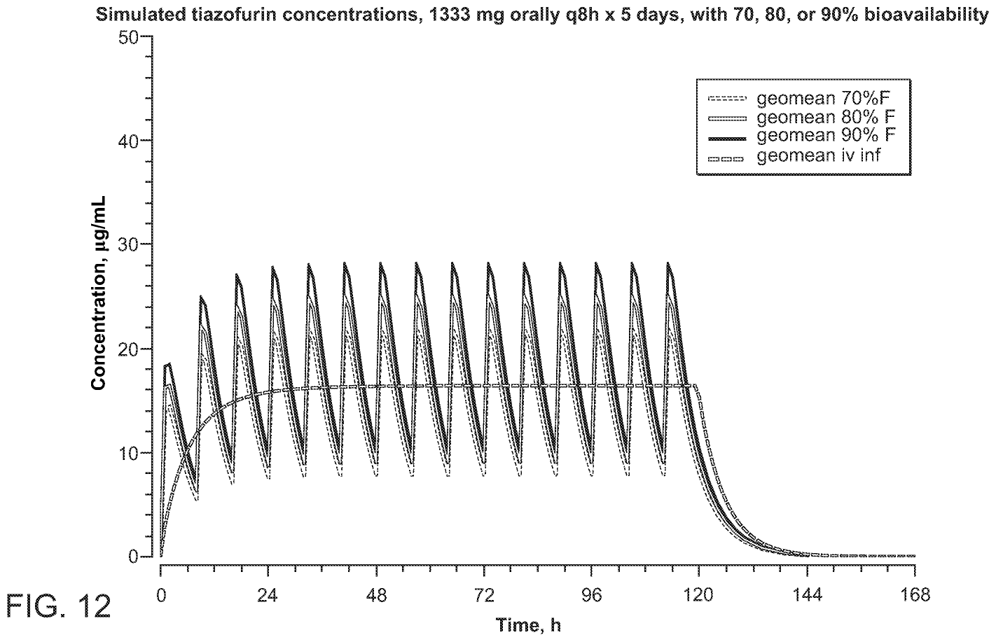

FIG. 12 is a graph of plasma tiazofurin levels in subjects after oral administration of a composition having 70%, 80%, or 90% bioavailability. Subjects are given 1333 mg tiazofurin orally once every 8 hours for five days. Continuous lines with dashed borders indicate geometric mean values following administration of a composition having 70%, bioavailability; continuous open lines indicate geometric mean values following administration of a composition having 80%, bioavailability; solid lines indicate geometric mean values following administration of a composition having 90%, bioavailability; and dashed lines indicate geometric mean values from control subjects given 4 g per day intravenously via continuous infusion for five days.

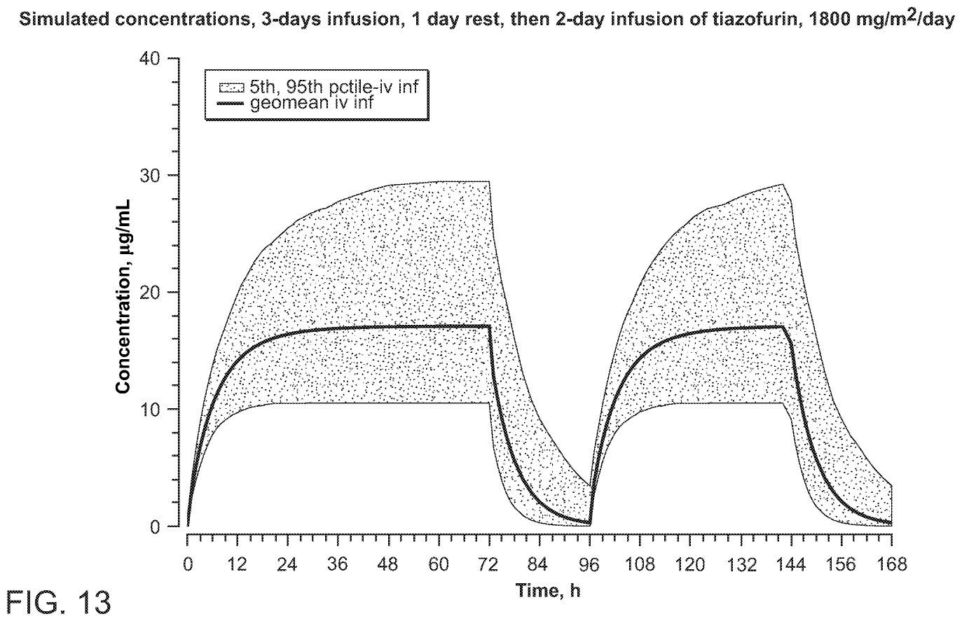

FIG. 13 is a graph of plasma tiazofurin levels in subjects after intravenous administration. Forty-year-old, 70-kg subjects having normal renal function are given 1800 mg/m² tiazofurin intravenously per day via continuous infusion for three days, given none for one day, and then given 1800 mg/m² tiazofurin intravenously per day via continuous infusion for two days. Dotted areas indicate values ranging from the 5th to 95th percentile; and solid lines indicate geometric mean values.

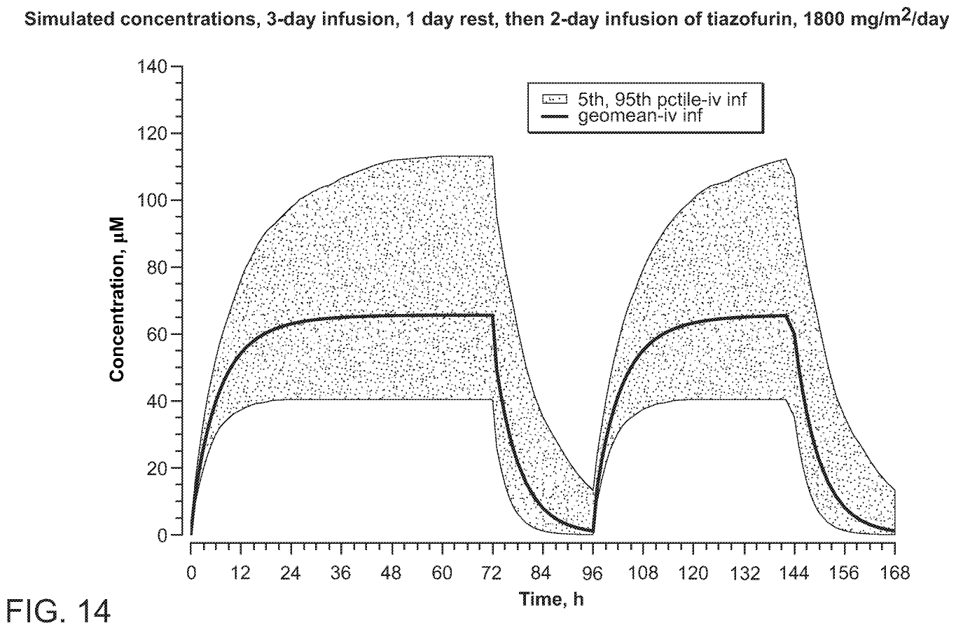

FIG. 14 is a graph of plasma tiazofurin levels in subjects after intravenous administration. Forty-year-old, 70-kg subjects having normal renal function are given 1800 mg/m² tiazofurin intravenously per day via continuous infusion for three days, given none for one day, and then given 1800 mg/m² tiazofurin intravenously per day via continuous infusion for two days. Dotted areas indicate values ranging from the 5th to 95th percentile; and solid lines indicate geometric mean values.

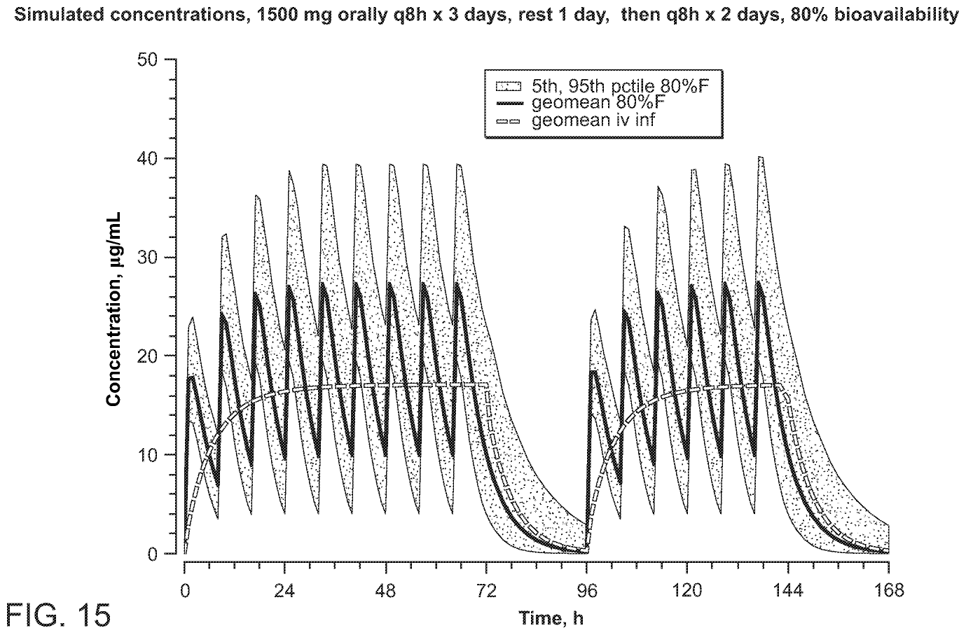

FIG. 15 is a graph of plasma tiazofurin levels (in micrograms/mL) in subjects after oral administration of a composition having 80% bioavailability. Forty-year-old, 70-kg subjects having normal renal function are given 1500 mg tiazofurin orally once every 8 hours for three days, given none for one day, and then given 1500 mg tiazofurin orally once every 8 hours for two days. Dotted areas indicate values ranging from the 5th to 95th percentile; solid lines indicate geometric mean values; and dashed lines indicate geometric mean values from control subjects given 4500 mg per day intravenously via continuous infusion for three days, given none for one day, and then given 4500 mg per day intravenously via continuous infusion for two days.

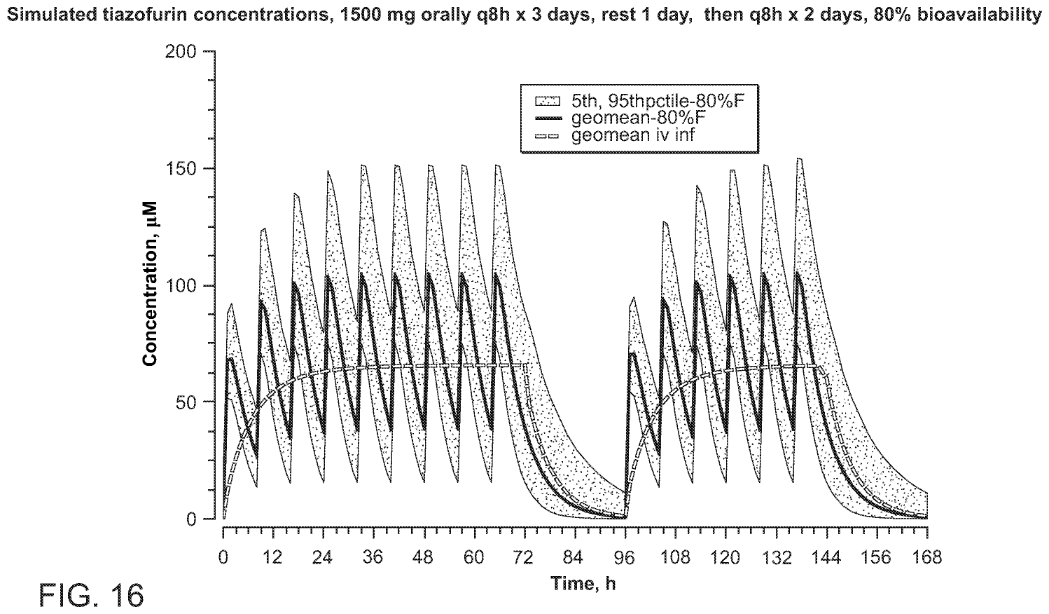

FIG. 16 is a graph of plasma tiazofurin levels (in micromoles/L) in subjects after oral administration of a composition having 80% bioavailability. Forty-year-old, 70-kg subjects having normal renal function are given 1500 mg tiazofurin orally once every 8 hours for three days, given none for one day, and then given 1500 mg tiazofurin orally once every 8 hours for two days. Dotted areas indicate values ranging from the 5th to 95th percentile; solid lines indicate geometric mean values; and dashed lines indicate geometric mean values from control subjects given 4500 mg per day intravenously via continuous infusion for three days, given none for one day, and then given 4500 mg per day intravenously via continuous infusion for two days.

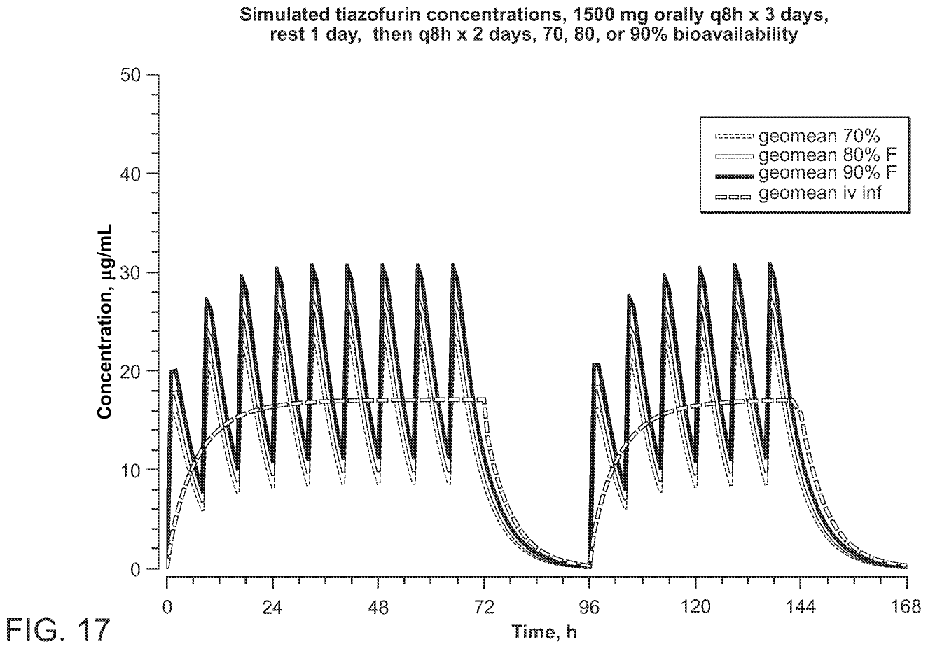

FIG. 17 is a graph of plasma tiazofurin levels in subjects after oral administration of compositions having 70%, 80%, or 90% bioavailability. Forty-year-old, 70-kg subjects having normal renal function are given 1500 mg tiazofurin orally once every 8 hours for three days, given none for one day, and then given 1500 mg tiazofurin orally once every 8 hours for two days. Continuous lines with dashed borders indicate geometric mean values following administration of a composition having 70%, bioavailability; continuous open lines indicate geometric mean values following administration of a composition having 80%, bioavailability; solid lines indicate geometric mean values following administration of a composition having 90%, bioavailability; and dashed open lines indicate geometric mean values from control subjects given 4500 mg per day intravenously via continuous infusion for three days, given none for one day, and then given 4500 mg per day intravenously via continuous infusion for two days.

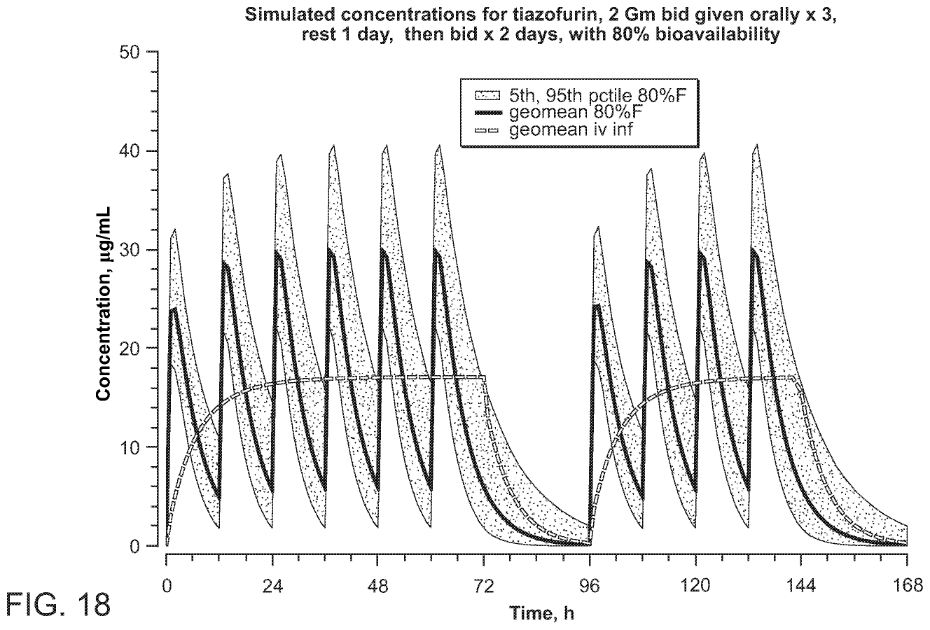

FIG. 18 is a graph of plasma tiazofurin levels in subjects after oral administration of a composition having 80% bioavailability. Forty-year-old, 70-kg subjects having normal renal function are given 2000 mg tiazofurin orally once every 12 hours for three days, given none for one day, and then given 2000 mg tiazofurin orally once every 12 hours for two days. Dotted areas indicate values ranging from the 5th to 95th percentile; solid lines indicate geometric mean values; and dashed lines indicate geometric mean values from control subjects given 4000 mg per day intravenously via continuous infusion for three days, given none for one day, and then given 4000 mg per day intravenously via continuous infusion for two days.

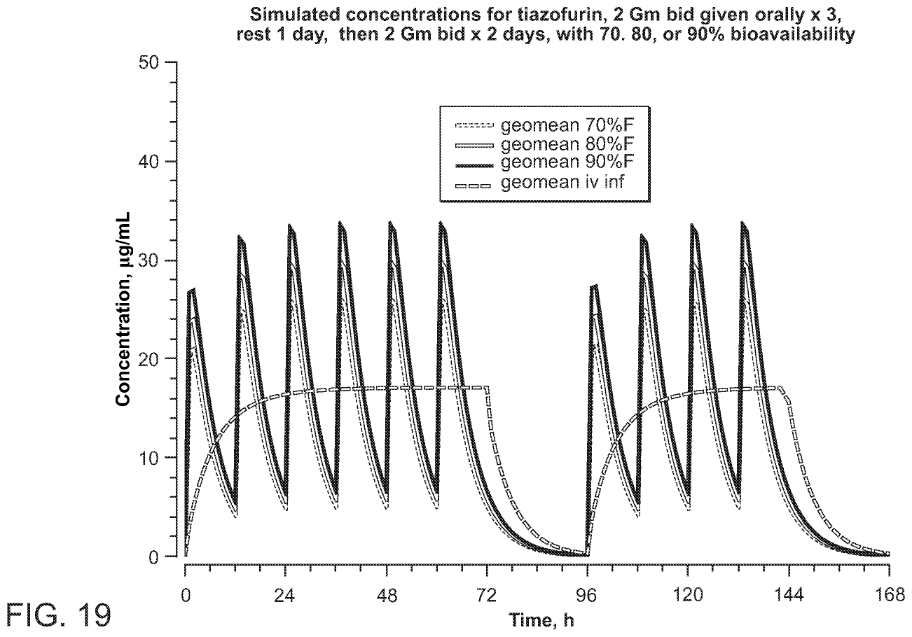

FIG. 19 is a graph of plasma tiazofurin levels in subjects after oral administration of compositions having 70%, 80%, or 90% bioavailability. Forty-year-old, 70-kg subjects having normal renal function are given 2000 mg tiazofurin orally once every 12 hours for three days, given none for one day, and then given 2000 mg tiazofurin orally once every 12 hours for two days. Continuous open lines with dashed border indicate geometric mean values following administration of a composition having 70%, bioavailability; continuous open lines indicate geometric mean values following administration of a composition having 80%, bioavailability; solid lines indicate geometric mean values following administration of a composition having 90%, bioavailability; and dashed open lines indicate geometric mean values from control subjects given 4000 mg per day intravenously via continuous infusion for three days, given none for one day, and then given 4000 mg per day intravenously via continuous infusion for two days.

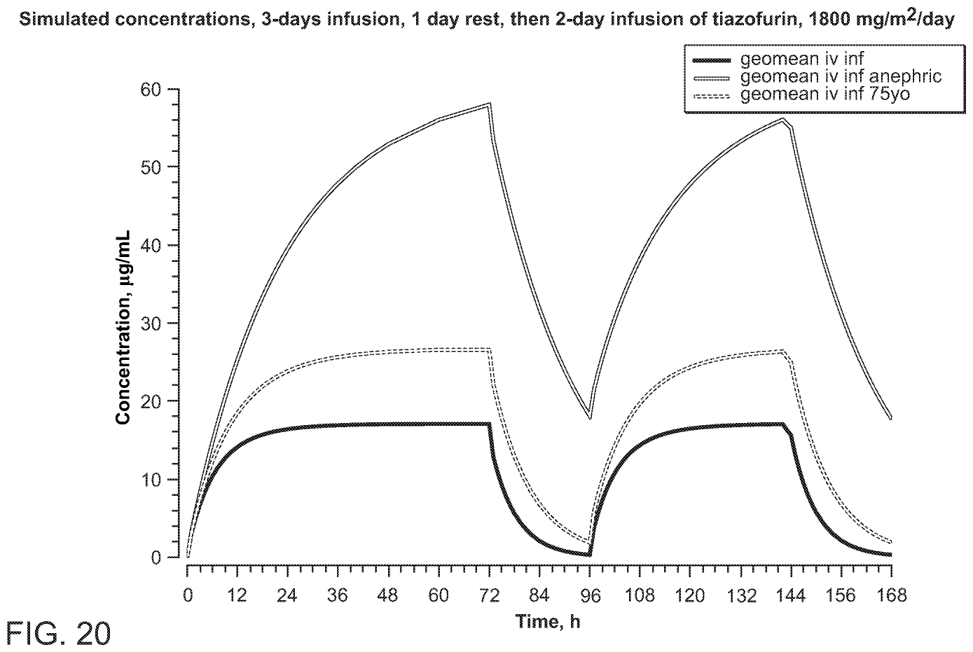

FIG. 20 is a graph of plasma tiazofurin levels in subjects after intravenous administration. Subjects are given 1800 mg/m² tiazofurin intravenously per day via continuous infusion for three days, given none for one day, and then given 1800 mg/m² tiazofurin intravenously per day via continuous infusion for two days. Solid lines indicate geometric mean values for 40-year-old, 70-kg subjects having normal renal function; continuous open lines indicate geometric mean values for 40-year-old, 70-kg, anephric subjects; and continuous open lines with dashed borders indicate geometric mean values for 75-year-old, 70-kg subjects with normal renal function.

In embodiments of the invention, the dose of an IMPDH inhibitor, such as tiazofurin, is adjusted to compensate for reduced renal function in the subject. Doses are determined based on the estimated clearance rate using the Cockcroft-Gault formula. Table 3 provides adjusted doses of tiazofurin needed to maintain equivalent exposure to subjects based on estimated creatinine clearance rates (CLcr).

TABLE 3

| Estimated CLcr | Estimated tiazofurin | Percent of standard |
|---|---|---|
| (mL/min) | CL (L/h) | tiazofurin dose |
| 0 | 2.14 | 28% |
| 20 | 3.06 | 40% |
| 40 | 3.98 | 52% |
| 60 | 4.90 | 64% |
| 80 | 5.82 | 75% |
| 100 | 6.74 | 90% |
| 120 | 7.65 | 100% |

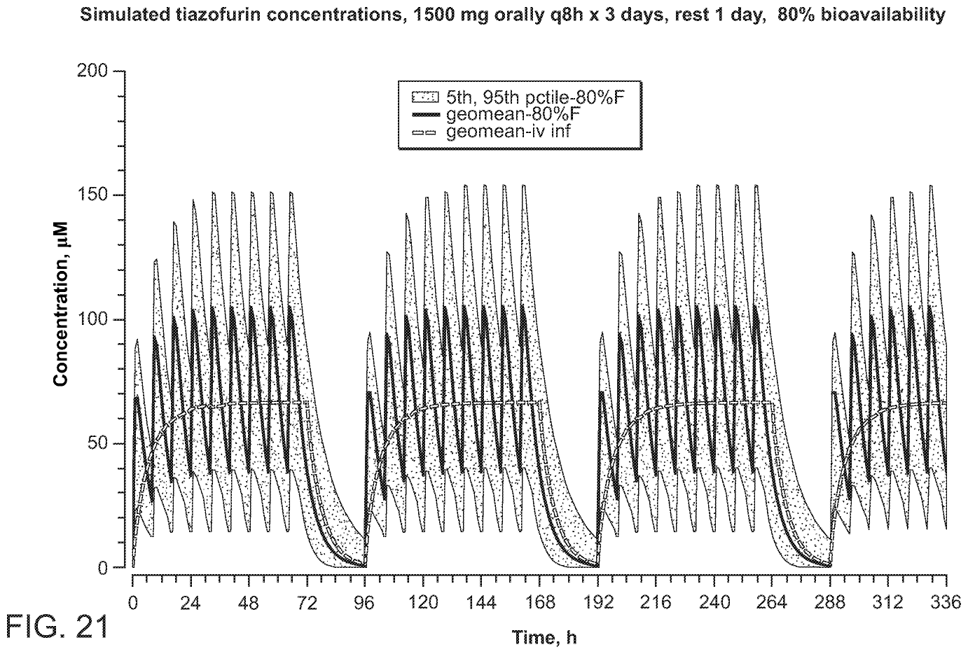

FIG. 21 is a graph of plasma tiazofurin levels in subjects after oral administration of a composition having 80% bioavailability. Forty-year-old, 70-kg subjects having normal renal function are given 1500 mg tiazofurin orally once every 8 hours for three days and then given none for one day to make a four-day cycle, which is repeated for a total of 14 days. Dotted areas indicate values ranging from the 5th to 95th percentile; solid lines indicate geometric mean values; and dashed lines indicate geometric mean values from control subjects given 4500 mg per day intravenously via continuous infusion for three days and then given none for one day to make a four-day cycle, which is repeated for a total of 14 days.

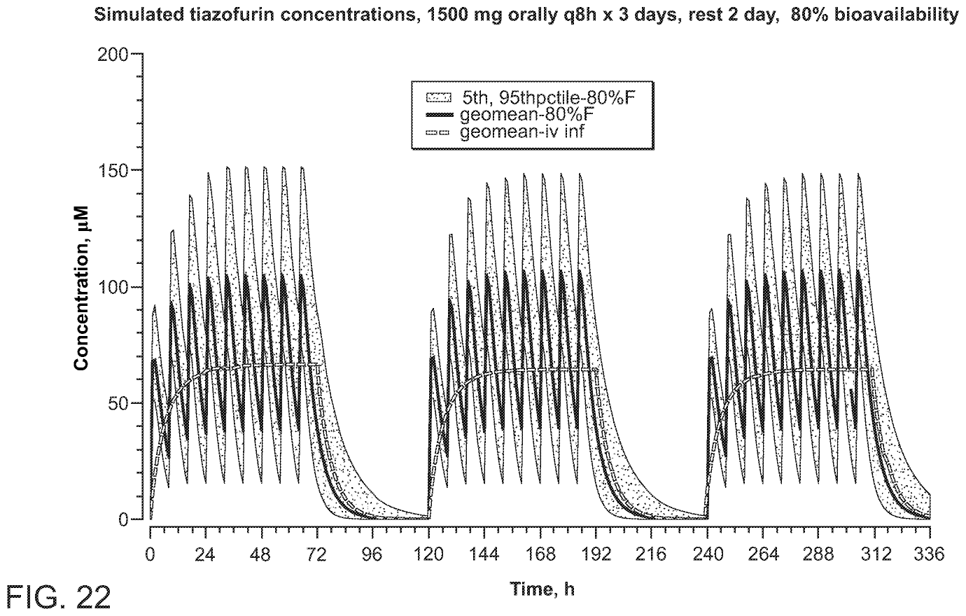

FIG. 22 is a graph of plasma tiazofurin levels in subjects after oral administration of a composition having 80% bioavailability. Forty-year-old, 70-kg subjects having normal renal function are given 1500 mg tiazofurin orally once every 8 hours for three days and then given none for two days to make a five-day cycle, which is repeated for a total of 14 days. Dotted areas indicate values ranging from the 5th to 95th percentile; solid lines indicate geometric mean values; and dashed lines indicate geometric mean values from control subjects given 4500 mg per day intravenously via continuous infusion for three days and then given none for one day to make a four-day cycle, which is repeated for a total of 14 days.

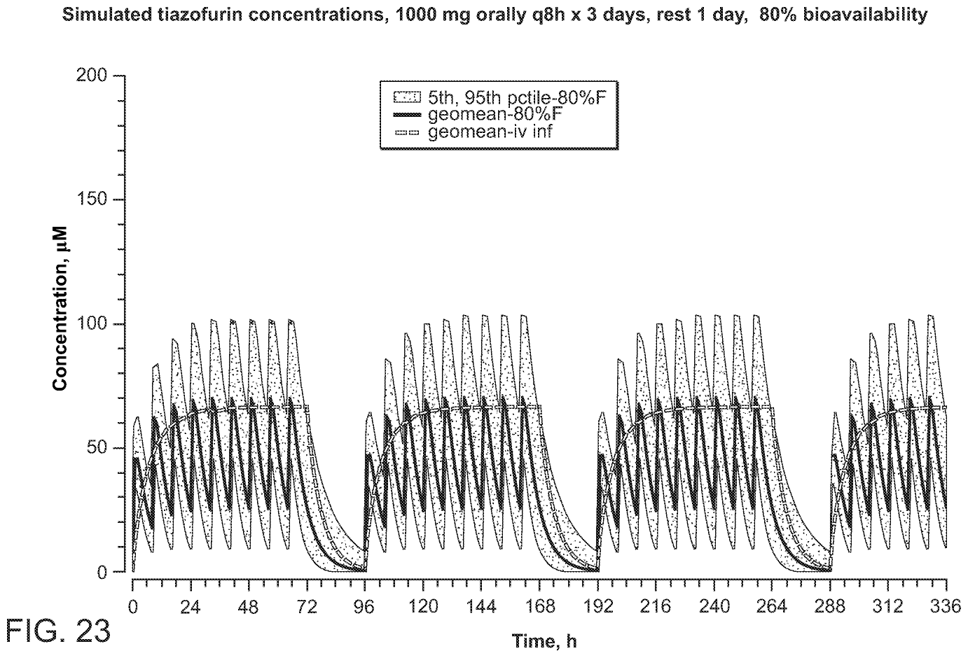

FIG. 23 is a graph of plasma tiazofurin levels in subjects after oral administration of a composition having 80% bioavailability. Forty-year-old, 70-kg subjects having normal renal function are given 1000 mg tiazofurin orally once every 8 hours for three days and then given none for one day to make a four-day cycle, which is repeated for a total of 14 days. Dotted areas indicate values ranging from the 5th to 95th percentile; solid lines indicate geometric mean values; and dashed lines indicate geometric mean values from control subjects given 3000 mg per day intravenously via continuous infusion for three days and then given none for one day to make a four-day cycle, which is repeated for a total of 14 days.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A composition comprising an inhibitor of inosine monophosphate dehydrogenase in a therapeutically effective amount that lowers or maintains in a subject a guanosine triphosphate (GTP) level below 10% of a reference GTP level for a period of at least 48 hours.

2. The composition of claim 1, wherein the reference GTP level is a level of GTP in the subject prior to administration of the composition to the subject.

3. The composition of claim 1, wherein the period is at least 72 hours.

4. The composition of claim 1, wherein the inhibitor is selected from the group consisting of mizoribine, mycophenolic acid, ribavirin, selenazofurin, taribavirin, and tiazofurin, wherein each of said inhibitors includes analogs, derivatives, prodrugs, micellar formulations, sustained release formulations, and salts thereof.

5. The composition of claim 4, wherein the inhibitor is tiazofurin or an analog, derivative, prodrug, micellar formulation, sustained release formulation, or salt thereof.

6. The composition of claim 1, wherein the composition is formulated for intravenous, oral, or parenteral administration.

7. The composition of claim 1, wherein the composition is formulated as divided dosages.

8. The composition of claim 1, wherein the composition is effective for treatment of leukemia.

9. An oral pharmaceutical composition comprising from about 10 mg to about 1,500 mg of tiazofurin or an analog, derivative, prodrug, micellar formulation, sustained release formulation, or salt thereof.

10. The composition of claim 9, wherein the composition comprises from about 100 mg to about 500 mg of tiazofurin or the analog, derivative, prodrug, micellar formulation, sustained release formulation, or salt thereof.

11. The composition of claim 9, wherein the composition is formulated as a single unit dosage.

12. A dosage comprising from about 0.2 grams to about 5 grams of orally deliverable tiazofurin or an analog, derivative, prodrug, micellar formulation, sustained release formulation, or salt thereof.

13. The dosage of claim 12, wherein the dosage comprises about 1 gram of orally deliverable tiazofurin or the analog, derivative, prodrug, micellar formulation, sustained release formulation, or salt thereof.

14. The dosage of claim 12, wherein the dosage is divided among multiple physical units.

15. The dosage of claim 12, wherein administration of the dosage to a subject multiple times in a 24-hour period provides tiazofurin or the analog, derivative, prodrug, micellar formulation, sustained release formulation, or salt thereof in a therapeutically effective amount that lowers or maintains in the subject a guanosine triphosphate (GTP) level below 10% of a reference GTP level for a period of at least 24 hours.

16. The dosage of claim 15, wherein administration of the dosage three times per 24-hour period provides tiazofurin or the analog, derivative, prodrug, micellar formulation, sustained release formulation, or salt thereof in the therapeutically effective amount.

17-20. (canceled)
